(19) United States
(12) Patent Application Publication
(10) Pub. No.: US 2005/0014807 A1
(43) Pub. Date: Jan. 20, 2005

(54) Therapeutic compounds for treating dyslipidemic conditions

(76) Inventors: Alan D Adams, Cranford, NJ (US); Shaei Y. Huang, Jeffersonville, PA (US); Bruno Tse, San Diego, CA (US); A. Brian Jones, Arkesden (UA)

Correspondence Address: MERCK AND CO INC, P O BOX 2000, RAHWAY, NJ, 070650907, US

(21) Appl. No.: 10/495,294

(22) PCT Filed: Nov. 18, 2002
(86) PCT No.: PCT/US02/36911

Related U.S. Application Data
(60) Provisional application No. 60/332,080, filed on Nov. 21, 2001.

(51) Int. Cl. C07D 261/20; A61K 31/42

(52) U.S. Cl. 514/379; 548/241

(57) ABSTRACT

Compounds of Formula I and the pharmaceutically acceptable salts and esters thereof, are novel LXR ligands and are useful in the treatment of dyslipidemic conditions, particularly depressed levels of HDL cholesterol.

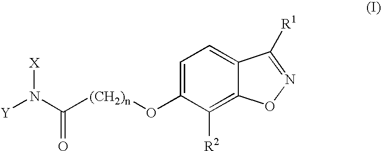

### BACKGROUND OF THE INVENTION

Recent publications in Nature Genetics, August 1999 (Young et al, page 316; Bodzioch et al, page 347; Brooks-Wilson et al, page 335, and Rust et al, page 352 ) showed that humans with mutations in the gene ABCA1 (also previously known in the art as ABC1) have low levels of high density lipoprotein (HDL). Low HDL levels are a risk factor for atherosclerosis, myocardial infarction and related conditions such as ischemic stroke. Therefore, increasing the expression of the ABCA1 gene would be expected to increase HDL levels and decrease the occurrence of atherosclerosis, myocardial infarction and related conditions such as ischemic stroke. It has been reported that expression of the ABCA1 gene is increased by cholesterol loading of cells (Langmann et al, Biochem. Biophys. Res. Comm., 257, 29-33 (1999)). LXRα is a nuclear receptor that is required for the induction of cholesterol 7α-hydroxylase in mouse liver following cholesterol feeding (Peet et al, Cell, 93, 693-704 (1998)). LXRα and LXRβ are activated by 22-(R)-hydroxycholesterol and other oxysterols (Janowski et al. Proc. Natl. Acad. Sci USA, 96, 266-271 (1999), Thomas A. Spencer et al. J. Med. Chem., 44, 886-897, (2001)). Some non-steroidal small molecule agonists of LXRα and LXRβ have been reported to affect circulating HDL levels, cholesterol absorption, reverse cholesterol transport and ABCA1 expression in vivo (J. R. Schultz, et al. Genes & Devel. 14, 2831-2838, (2000), J. J. Repa et al. Science, 289, 1524-1529, (2000)) It has been found that LXRα and/or LXRβ cause the induction or regulation of ABCA1 expression, and that small molecule ligands of LXR are useful as drugs to increase the expression of ABCA1, increase levels of HDL and thereby decrease the risk of atherosclerosis, myocardial infarction and related conditions such as peripheral vascular disease and ischemic stroke.

The various dyslipidemic conditions, which are risk factors for atherosclerosis, are currently treated with several different classes of drugs, such as statins which are HMG-CoA reductase inhibitors, bile acid sequestrants (e.g., cholestyramine and colestipol), nicotinic acid (niacin), and fibrates. However, except for niacin, most of these treatments do not raise HDL as their primary effect. With favorable outcomes in many human studies, the statin class of drugs is used to modulate LDL and, to a lesser extent, HDL and triglycerides. Conditions principally characterized by elevated plasma triglycerides and low HDL are frequently treated with drugs belonging to the fibrate class. The fibrates are PPAR alpha agonists that lower triglycerides and raise HDL in many instances. There are no currently marketed drugs whose principal actions are mediated by LXR.

We have now discovered a new class of small molecules which are LXR ligands, i.e., LXRα and/or LXRβ ligands, and are therefore expected to be useful for modulation of HDL levels, ABCA1 gene expression and reverse cholesterol transport. The instant compounds have been shown to raise plasma levels of HDL in animal models and to increase cholesterol efflux from cells in vitro. These biological activities are critical for reverse cholesterol transport.

The novel compounds of this invention are intended as a treatment for dyslipidemias, especially low plasma HDL cholesterol levels, as well as for treatment and/or prevention of lipid accumulation in atherosclerotic plaques, which is an underlying cause or aggravating factor in atherosclerosis.

### SUMMARY OF THE INVENTION

Compounds of Formula I are useful in the treatment of dyslipidemic conditions including below-desirable levels of HDL cholesterol.

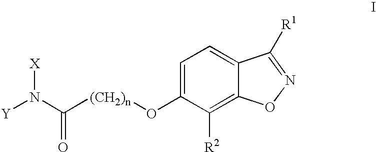

One object of the instant invention is to provide a method for treating depressed plasma HDL cholesterol levels comprising administering a therapeutically effective amount of a compound of Formula I to a patient in need of such treatment.

Another object is to provide a method for preventing or treating dyslipidemic conditions comprising administering a prophylactically or therapeutically effective amount, as appropriate, of a compound of Formula I to a patient in need of such treatment.

As a further object, methods are provided for preventing or reducing the risk of developing atherosclerosis, as well as for halting or slowing the progression of atherosclerotic disease once it has become clinically evident, comprising the administration of a prophylactically or therapeutically effective amount, as appropriate, of a compound of Formula I to a patient who is at risk of developing atherosclerosis or who already has atherosclerotic disease. The method of this invention also serves to remove cholesterol from tissue deposits such as xanthomas and atherosclerotic lesions by hastening the efflux of cholesterol from cells in those lesions. Additional objects will be evident from the following detailed description.

Other objects of this invention are to provide processes for making the compounds of Formula I and to provide novel pharmaceutical compositions comprising these compounds. Additional objects will be evident from the following detailed description.

### DETAILED DESCRIPTION OF THE INVENTION

The invention includes compounds having the formula 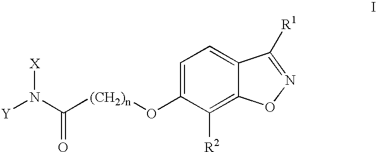
or a pharmaceutically acceptable salt thereof, wherein
R¹ is selected from the group consisting of:
   (a) —CF₃,
   (b) —C₁₋₆ alkyl, and
   (c) —(CH₂)₀₋₂-phenyl;
R² is selected from the group consisting of:
   (a) —C₁₋₆ alkyl,
   (b) —COOR³,
   (c) —CR³R⁴—O—R⁵,
   (d) —CR³R⁴—S—R⁵ and
   (e) —COR³;
R³, R⁴ and R⁵ are independently selected at each occurrence from the group consisting of —H, phenyl and C₁₋₆ alkyl;
n is an integer selected from 2, 3, 4, 5 and 6;
X is selected from the group consisting of:.
   (a) —H and
   (b) —C₁₋₆ alkyl;
Y is selected from the group consisting of:
   (a) —H,
   (b) —C₁₋₆ alkyl unsubstituted or substituted with a substituent selected from the group consisting of:
      (i) —COOR⁶,
      (ii) phenyl, unsubstituted or substituted with —COOR⁶, and
      (iii) furanyl,
   (c) thiophenyl, unsubstituted or substituted with —COOR⁶, and
   (d) pyridinyl, unsubstituted, monosubstituted with a substituent selected from the group consisting of C₁₋₃ alkyl and halogen, or independently disubstituted with two substituents selected from the group consisting of C₁₋₃ alkyl and halogen,
   where R⁶ is selected from the group consisting of —H, phenyl and C₁₋₆ alkyl; or Y and X are joined together with the nitrogen to which they are attached to form a piperidinyl ring.

In a class of compounds of the invention, and pharmaceutically acceptable salts thereof, R¹ is selected from the group consisting of CF₃ and C₁₋₆ alkyl, R² is C₁₋₆ alkyl, and n is 3.

In a subclass of the class of compounds, and pharmaceutically acceptable salts thereof,
X is selected from the group consisting of H and C₁₋₃ alkyl, and
Y is selected from the group consisting of:
   (a) —H,
   (b) —C₁₋₆ alkyl unsubstituted or substituted with a substituent selected from the group consisting of:
      (i) —COOR⁶,
      (ii) phenyl, unsubstituted or substituted with —COOR⁶, and
      (iii) furanyl,
   (c) thiophenyl, unsubstituted or substituted with —COOR⁶, and
   (d) pyridinyl, unsubstituted, monosubstituted with a substituent selected from the group consisting of C₁₋₃ alkyl and halogen, or independently disubstituted with two substituents selected from the group consisting of C₁₋₃ alkyl and halogen,
   where R⁶ is selected from the group consisting of —H, phenyl and C₁₋₆ alkyl; or Y and X are joined together with the nitrogen to which they are attached to form a piperidinyl ring.

In a group of the subclass of compounds, and pharmaceutically acceptable salts thereof, R¹ is selected from the group consisting of CF₃ and —CH₂C(CH₃)₃, R² is —CH₂CH₂CH₃, X is selected from the group consisting of H and —CH₃, Y is selected from the group consisting of 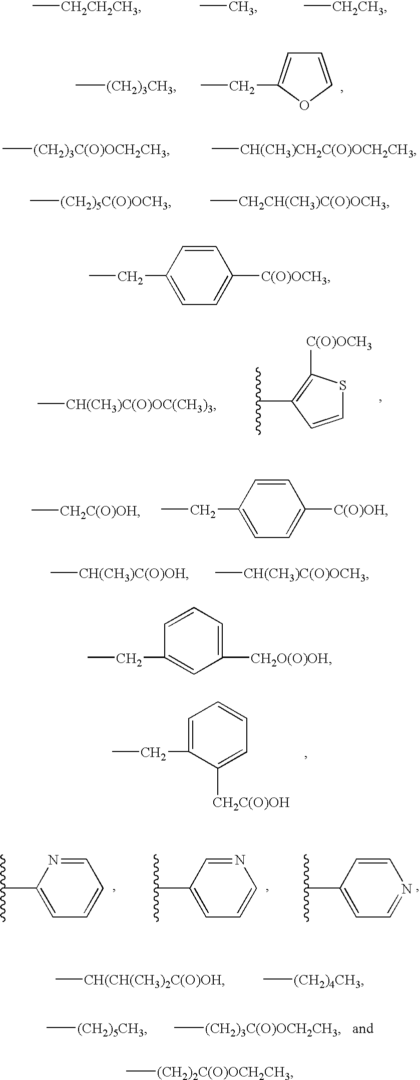
or Y and X are joined together with the nitrogen to which they are attached to form a piperidinyl ring.

Examples of the invention have the following particular structures: 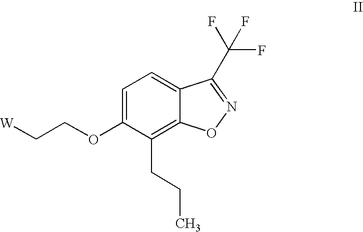
where W is

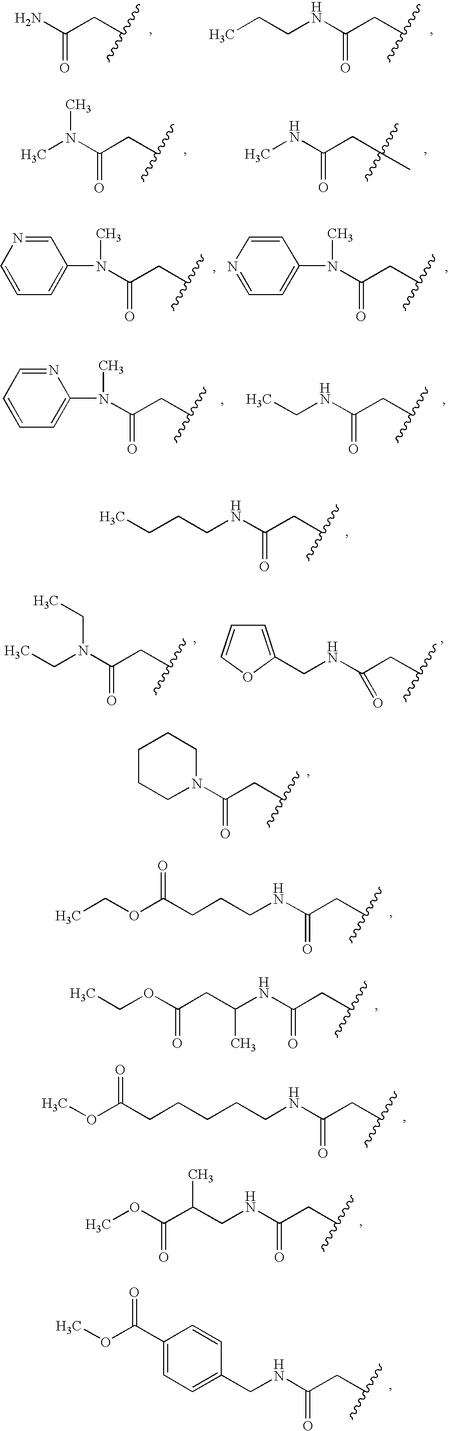

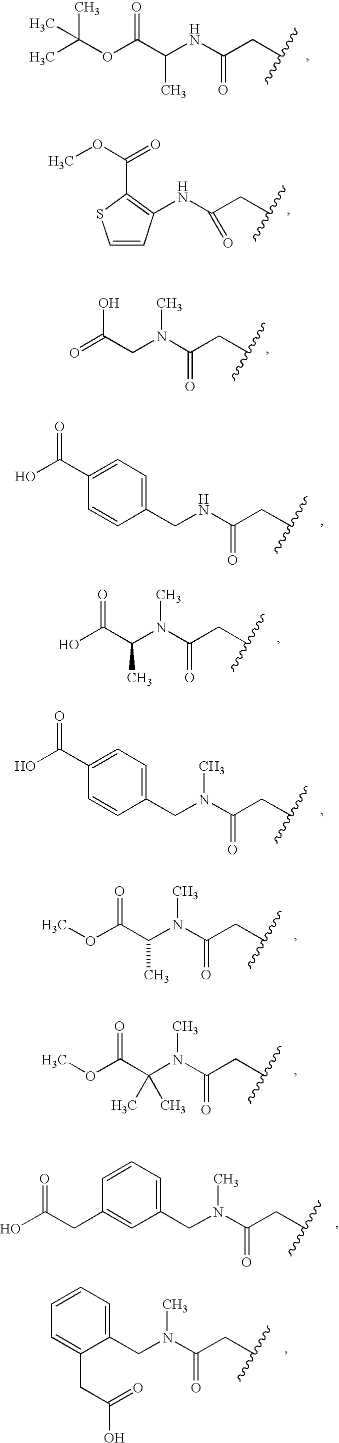

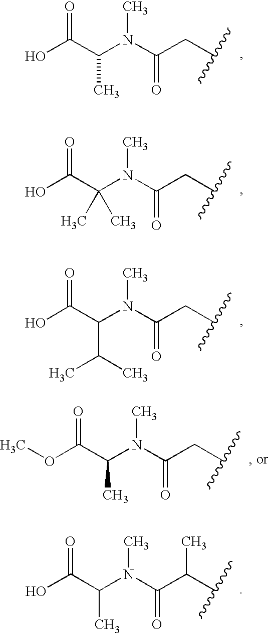

Additional examples have the following particular structures: 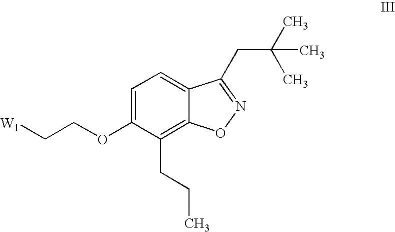
where W₁is

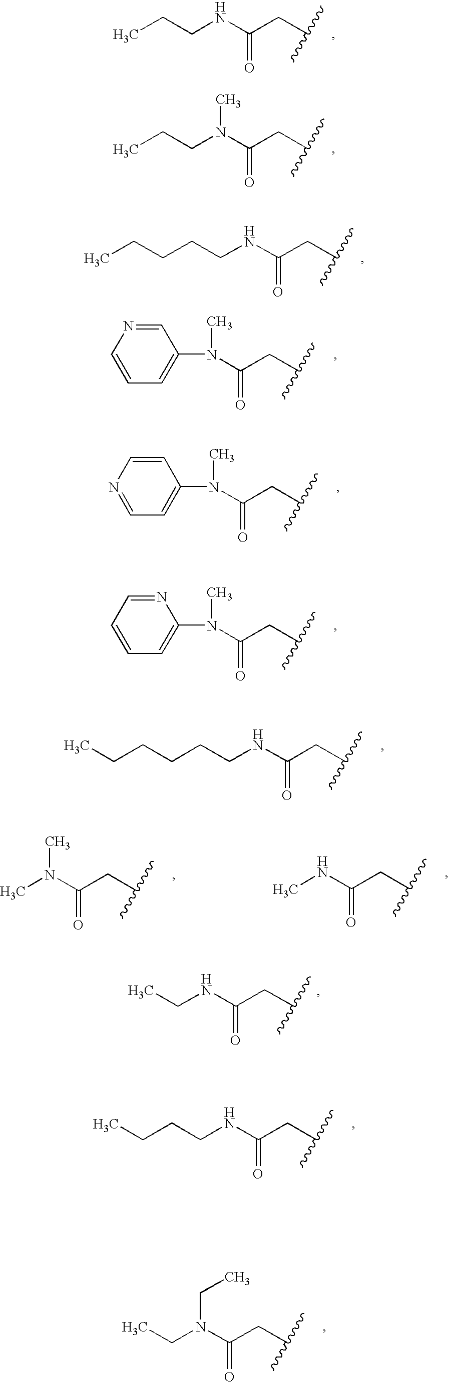

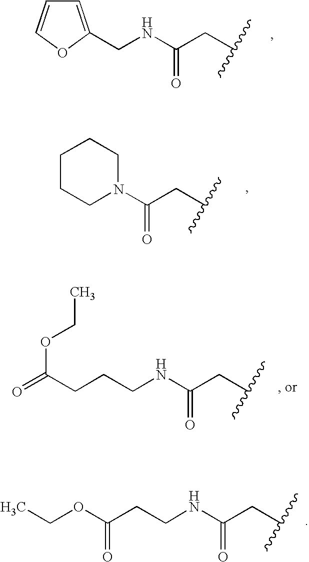

A preferred group of examples includes the following compounds: 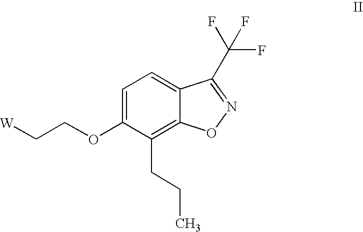
where W is 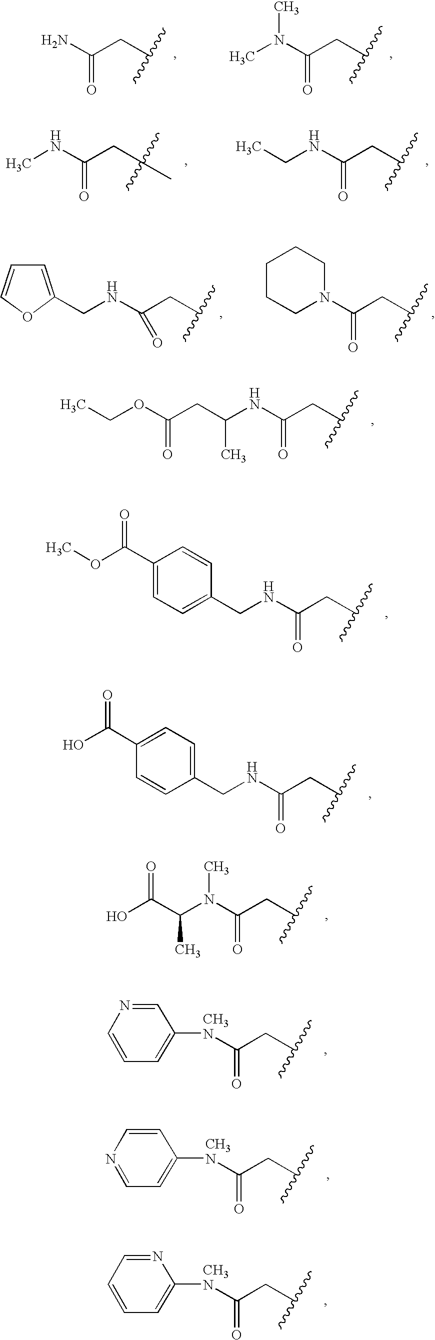 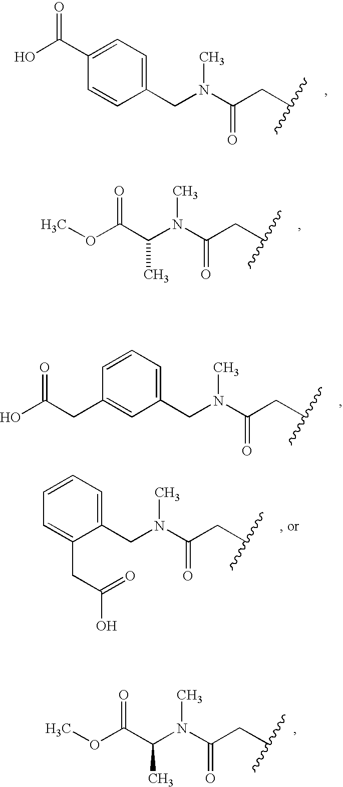
and 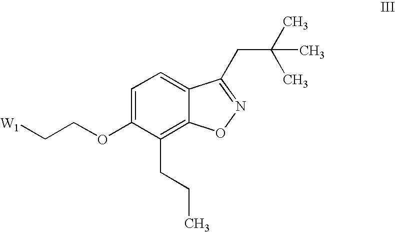
where W₁ is

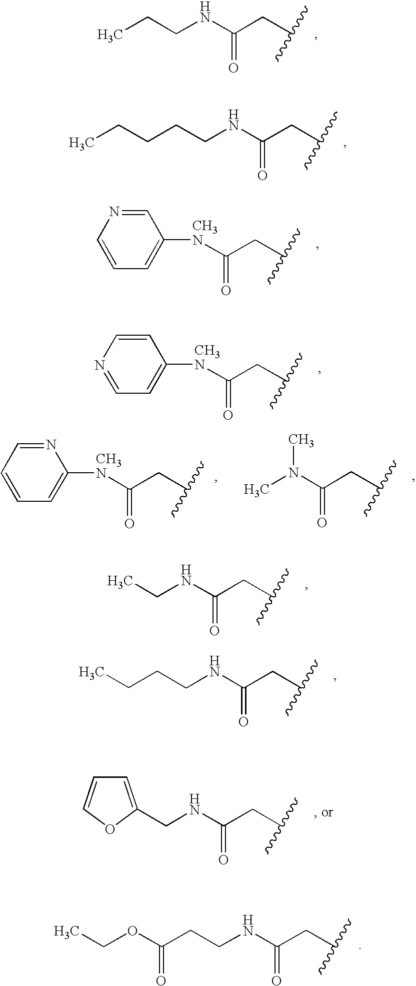

A more preferred group of examples includes the following compounds: 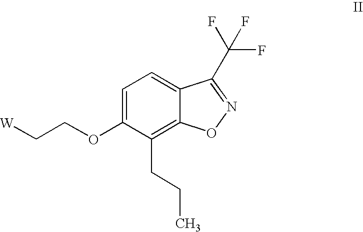
where W is 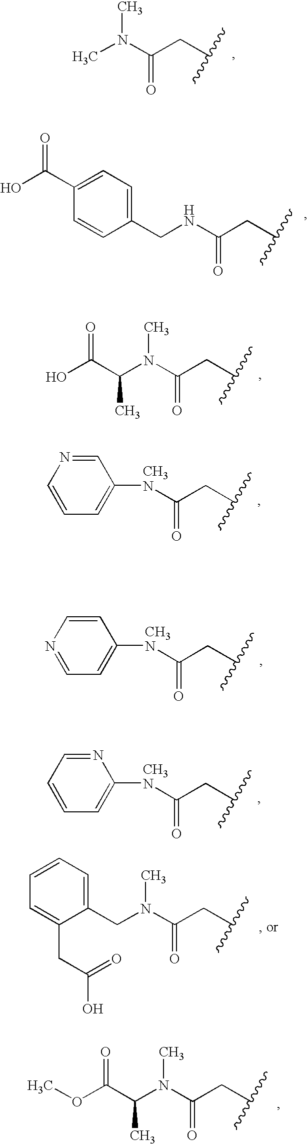
and 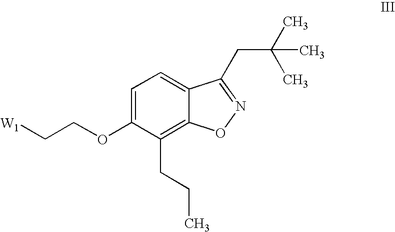
where W₁ is

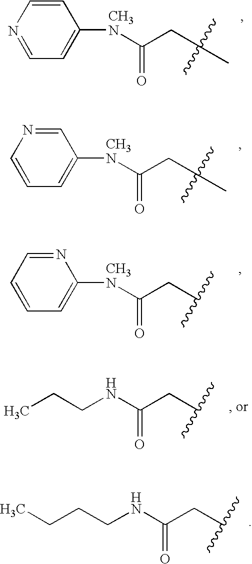

Compounds of the invention are LXR ligands, including agonists and antagonists, which are useful for modulating HDL levels.

As used herein “alkyl” is intended to include both branched- and straight-chain saturated aliphatic hydrocarbon groups having the specified number of carbon atoms, e.g., methyl (Me), ethyl (Et), n-propyl (Pr), n-butyl (Bu), n-pentyl, n-hexyl, and the isomers thereof such as isopropyl (i-Pr), isobutyl (i-Bu), secbutyl (s-Bu), tertbutyl (t-Bu), isopentyl, isohexyl and the like. Alkyl groups are unsubstituted or optionally substituted where noted herein. As intended herein, an unsubstituted branched or straight chain alkyl group has the general formula CₙH₂ₙ₊₁, for example CH₃CH₂—, (CH₃)₂CH—, CH₃—C(CH₃)₂—CH₂— and the like. The base alkyl portion of a mono-substituted branched or straight chain alkyl group has the general formula CₙH₂ₙ, for example —CH₂CH₂—, 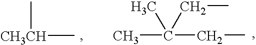
—CH₂C(CH₃)₂CH₂—, —CH₂—C(CH₃)(CH₂CH₃)—CH₂—, and the like. The base alkyl portion of a di-substituted branched or straight chain alkyl group has the general formula CₙH₂ₙ₋₁, for example 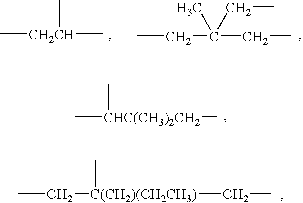
and the like. Alkyl groups with additional substitutions follow this continuing pattern.

The term halo or halogen is meant to include fluoro, chloro, bromo and iodo, unless otherwise noted. Fluoro is preferred.

When referring to moieties which may optionally be substituted herein, e.g., alkyl groups, phenyl groups and the like, the phrase used herein “independently unsubstituted or substituted with a substituent independently selected at each occurrence” is intended to mean that each carbon atom that is available for substitution in the given moiety may independently be unsubstituted or substituted, and substituted atoms may have one or more substituents that are the same or different which results in the creation of a stable structure. Particularly, optionally substituted moieties defined within Formula I are unsubstituted or each moiety has one or two substituents, and each substituted carbon atom within the moiety is mono- or di-substituted. More particularly, optionally substituted moieties defined within Formula I are unsubstituted or have one substituent.

The compounds of the present invention may be chiral and the present compounds may occur as diasteriomeric mixtures, racemates (racemic mixtures) and as individual diasteriomers or enantiomers with all such isomeric forms being included within the scope of this invention, except where the stereoconfiguration of a specific chiral center is defined or depicted otherwise. Furthermore, some of the crystalline forms for compounds of the present invention may exist as polymorphs and as such are intended to be included in the present invention. In addition, some of the compounds of the instant invention may form solvates with water or common organic solvents. Such solvates and hydrates are encompassed within the scope of this invention.

### ABBREVIATIONS

Some abbreviations used herein are as follows: Ac is acetyl [CH₃C(O)—]; PG is protecting group; Ph is phenyl; PhMe is toluene; Bn is benzyl; BnBr is benzylbromide; MeOH is methanol; DMF is N,N-dimethylformamide; DMSO is di-methyl sulfoxide; THF is tetrahydrofuran; TMS is trimethylsilyl; HOBt is 1-hydroxybenzotriazole; EDAC (or EDC) is 1-ethyl-3-[3-(dimethylamino)propyl]carbodiimide HCl; NaHMDS is sodium hexamethyldisiliazide; DIBAL is diisobutylaluminum hydride; TPAP is tetrapropylammonium perruthenate; NMO is N-methylmorpholine N oxide; HPLC is high performance liquid chromatography; TLC is thin layer chromatography; RT is ambient temperature.

In this specification, methyl substituents may be represented by

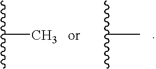

For example, the structures 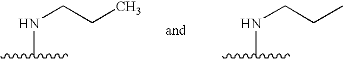
have equivalent meanings.

### GENERAL SCHEMES

The compounds of this invention can be prepared employing the following general procedures. Benzisoxazole intermediates may be prepared from commercially available or readily accessible resorcinols as shown in scheme I or alternate synthetic pathways as reported in the literature. See for example; Shutske, G. M.; et al.; J Med Chem, 25 (1), 36, (1982), Poissonnet, G. Synth Commun, 27 (22), 3839-3846, (1997), Crabbe, P.; Villarino, A.; Muchowski, J. M.; J Chem Soc, Perkin Trans 1, 1973, 2220. 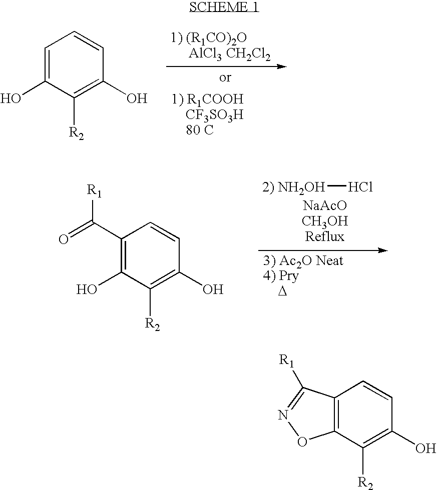
Elaboration of the benzisoxazole fragments by appending a carboxylic acid residue connected by an alkyl tether is readily accomplished. One method illustrated in Scheme 2 is alkylation of the free phenol with a haloalkyl residue carrying any of a host of functional groups convertible to a carboxylic acid. Hydroxy alkyl residues carrying a substituent convertible to a carboxylic acid may also be coupled to the phenol by any of several single or multiple step sequences. An example of a method to append a hydroxy alkyl residue would be the Mitsunobu coupling of a primary alcohol in the presence of DIAD and triphenylphosphine. 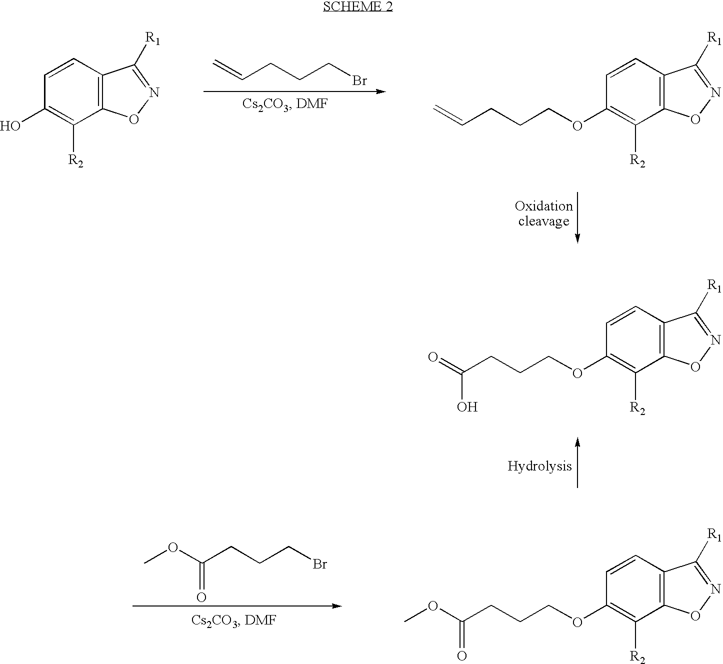
Various protected forms of the carboxylic acid residue are deprotected, as for esters, or unmasked, as for the vinyl residue.

A host of methods are available and well known in the literature to facilitate the conversion of the carboxylic acid residue to the amide derivatives describe here. Well known examples include the peptide coupling reagents DCC, EDC and CDI.

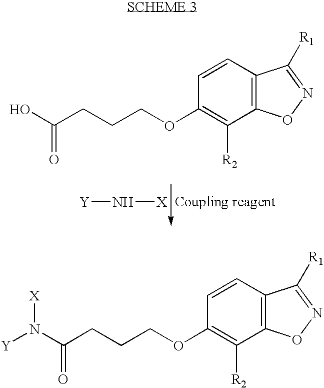

The instant invention provides methods for treating lipid disorders, particularly for treating below-desired plasma HDL cholesterol levels, as well as for treating and/or reducing the risk for diseases and conditions affected by LXR activity, comprising administering a therapeutically effective amount of a compound of Formula I to a person in need of such treatment. Any patient having a depressed plasma HDL cholesterol level, or desiring to increase their HDL cholesterol level may use this treatment. Particularly suitable patients in need of such treatment are those whose plasma HDL cholesterol level is depressed, i.e., below the clinically desirable level. Currently, the clinically desirable HDL cholesterol level is considered to be about 40 mg/dl or higher in men and about 50 mg/dl or higher in women.

The method of this invention also serves to prevent lipid accumulation in, or remove lipids from, tissue deposits such as atherosclerotic plaques or xanthomas in a patient with atherosclerotic disease manifest by clinical signs such as angina, claudication, bruits, one that has suffered a myocardial infarction or transient ischemic attack, or one diagnosed by angiography, sonography or MRI.

Further provided are methods for preventing or reducing the risk of developing atherosclerosis, as well as for halting or slowing the progression of atherosclerotic disease once it has become clinically evident, comprising the administration of a prophylactically or therapeutically effective amount, as appropriate, of a compound of Formula I to a mammal, including a human, who is at risk of developing atherosclerosis or who already has atherosclerotic disease.

Atherosclerosis encompasses vascular diseases and conditions that are recognized and understood by physicians practicing in the relevant fields of medicine. Atherosclerotic cardiovascular disease including restenosis following revascularization procedures, coronary heart disease (also known as coronary artery disease or ischemic heart disease), cerebrovascular disease including multi-infarct dementia, and peripheral vessel disease including erectile dysfunction are all clinical manifestations of atherosclerosis and are therefore encompassed by the terms “atherosclerosis” and “atherosclerotic disease.”

A compound of Formula I may be administered to prevent or reduce the risk of occurrence, or recurrence where the potential exists, of a coronary heart disease event, a cerebrovascular event, and/or intermittent claudication. Coronary heart disease events are intended to include CHD death, myocardial infarction (i.e., a heart attack), and coronary revascularization procedures. Cerebrovascular events are intended to include ischemic or hemorrhagic stroke (also known as cerebrovascular accidents) and transient ischemic attacks. Intermittent claudication is a clinical manifestation of peripheral vessel disease. The term “atherosclerotic disease event” as used herein is intended to encompass coronary heart disease events, cerebrovascular events, and intermittent claudication. It is intended that persons who have previously experienced one or more non-fatal atherosclerotic disease events are those for whom the potential for recurrence of such an event exists.

Accordingly, the instant invention also provides a method for preventing or reducing the risk of a first or subsequent occurrence of an atherosclerotic disease event comprising the administration of a prophylactically effective amount of a compound of Formula I to a patient at risk for such an event. The patient may or may not have atherosclerotic disease at the time of administration, or may be at risk for developing it.

Persons to be treated with the instant therapy include those with dyslipidemic conditions including depressed or below-desirable plasma levels of HDL cholesterol, as well as those at risk of developing atherosclerotic disease and of having an atherosclerotic disease event. Standard atherosclerotic disease risk factors are known to the average physician practicing in the relevant fields of medicine. Such known risk factors include but are not limited to hypertension, smoking, diabetes, low levels of high density lipoprotein cholesterol, and a family history of atherosclerotic cardiovascular disease. Published guidelines for determining those who are at risk of developing atherosclerotic disease can be found in: National Cholesterol Education Program, Second report of the Expert Panel on Detection, Evaluation, and Treatment of High Blood Cholesterol in Adults (Adult Treatment Panel II), National Institute of Health, National Heart Lung and Blood Institute, NIH Publication No. 93-3095, September 1993; abbreviated version: Expert Panel on Detection, Evaluation, and Treatment of High Blood Cholesterol in Adults, Summary of the second report of the national cholesterol education program (NCEP) Expert Panel on Detection, Evaluation, and Treatment of High Blood Cholesterol in Adults (Adult Treatment Panel II), JAMA, 1993, 269, pp. 3015-23. People who are identified as having one or more of the above-noted risk factors are intended to be included in the group of people considered at risk for developing atherosclerotic disease. People identified as having one or more of the above-noted risk factors, as well as people who already have atherosclerosis, are intended to be included within the group of people considered to be at risk for having an atherosclerotic disease event.

The term “patient” includes mammals, especially humans, who use the instant active agents for the prevention or treatment of a medical condition. Administering of the drug to the patient includes both self-administration and administration to the patient by another person. The patient may be in need of treatment for an existing disease or medical condition, or may desire prophylactic treatment to prevent or reduce the risk for diseases and medical conditions affected by HDL cholesterol.

The term “therapeutically effective amount” is intended to mean that amount of a drug or pharmaceutical agent that will elicit the biological or medical response of a tissue, a system, animal or human that is being sought by a researcher, veterinarian, medical doctor or other clinician. The term “prophylactically effective amount” is intended to mean that amount of a pharmaceutical drug that will prevent or reduce the risk of occurrence of the biological or medical event that is sought to be prevented in a tissue, a system, animal or human by a researcher, veterinarian, medical doctor or other clinician. Particularly, the dosage amount of a compound of Formula I that a patient receives can be selected so as to achieve the amount of lipid level modification desired, particularly to achieve a desired level of HDL cholesterol. The dosage a patient receives may also be titrated over time in order to reach a target lipid profile. The dosage regimen utilizing a compound of Formula I is selected in accordance with a variety of factors including type, species, age, weight, sex and medical condition of the patient; the severity of the condition to be treated; the potency of the compound chosen to be administered; drug combinations; the route of administration; and the renal and hepatic function of the patient. A consideration of these factors is well within the purview of the ordinarily skilled clinician for the purpose of determining the therapeutically effective or prophylactically effective dosage amount needed to prevent, counter, or arrest the progress of the condition.

An effective amount of compound for use in the method of this invention is about 0.01 mg/kg to about 140 mg/kg of body weight per day, or about 0.5 mg to about 7 g per patient in single or divided doses per day. More particularly, an amount of about 0.5 mg to about 3.5 g per patient, e.g. 0.5, 1.0, 1.5, 2.0, 2.5, 3.0 and 3.5 mg, in single or divided doses per day can be administered. However, dosage amounts will vary depending on factors as noted above, including the potency of the particular compound. Although the active drug of the present invention may be administered in divided doses, for example from one to four times daily, a single daily dose of the active drug is preferred.

The active drug employed in the instant therapy can be administered in such oral forms as tablets, capsules, pills, powders, granules, elixirs, tinctures, suspensions, syrups, and emulsions. Oral formulations are preferred.

Administration of the active drug can be via any pharmaceutically acceptable route and in any pharmaceutically acceptable dosage form. This includes the use of oral conventional rapid-release, time controlled-release and delayed-release (such as enteric coated) pharmaceutical dosage forms. Additional suitable pharmaceutical compositions for use with the present invention are known to those of ordinary skill in the pharmaceutical arts; for example, see Remington's Pharmaceutical Sciences, Mack Publishing Co., Easton, Pa.

In the methods of the present invention, the active drug is typically administered in admixture with suitable pharmaceutical diluents, excipients or carriers (collectively referred to herein as “carrier” materials) suitably selected with respect to the intended form of administration, that is, oral tablets, capsules, elixirs, syrups and the like, and consistent with conventional pharmaceutical practices.

For instance, for oral administration in the form of a tablet or capsule, the active drug component can be combined with a non-toxic, pharmaceutically acceptable, inert carrier such as lactose, starch, sucrose, glucose, modified sugars, modified starches, methyl cellulose and its derivatives, dicalcium phosphate, calcium sulfate, mannitol, sorbitol and other reducing and non-reducing sugars, magnesium stearate, steric acid, sodium stearyl fumarate, glyceryl behenate, calcium stearate and the like. For oral administration in liquid form, the drug components can be combined with non-toxic, pharmaceutically acceptable inert carrier such as ethanol, glycerol, water and the like. Moreover, when desired or necessary, suitable binders, lubricants, disintegrating agents and coloring and flavoring agents can also be incorporated into the mixture. Stabilizing agents such as antioxidants, for example butylated hydroxyanisole (BHA), 2,6-di-tert-butyl-4-methylphenol (BHT), propyl gallate, sodium ascorbate, citric acid, calcium metabisulphite, hydroquinone, and 7-hydroxycoumarin, can also be added to stabilize the dosage forms. Other suitable components include gelatin, sweeteners, natural and synthetic gums such as acacia, tragacanth or alginates, carboxymethylcellulose, polyethylene glycol, waxes and the like.

The active drug can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

Active drug may also be delivered by the use of monoclonal antibodies as individual carriers to which the compound molecules are coupled. Active drug may also be coupled with soluble polymers as targetable drug carriers. Such polymers can include polyvinyl-pyrrolidone, pyran copolymer, polyhydroxy-propyl-methacrylamide-phenol, polyhydroxy-ethyl-aspartamide-phenol, or polyethyleneoxide-polylysine substituted with palmitoyl residues. Furthermore, active drug may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and cross linked or amphipathic block copolymers of hydrogels.

The instant invention also encompasses a process for preparing a pharmaceutical composition comprising combining a compound of Formula I with a pharmaceutically acceptable carrier. Also encompassed is the pharmaceutical composition which is made by combining a compound of Formula I with a pharmaceutically acceptable carrier.

In a broad embodiment, any suitable additional active agent or agents may be used in combination with the compound of Formula I in a single dosage formulation, or may be administered to the patient in a separate dosage formulation, which allows for concurrent or sequential administration of the active agents. One or more additional active agents may be administered with a compound of Formula I. The additional active agent or agents can be lipid modifying compounds or agents having other pharmaceutical activities, or agents that have both lipid-modifying effects and other pharmaceutical activities. Examples of additional active agents which may be employed include but are not limited to HMG-CoA reductase inhibitors, which include statins in their lactonized or dihydroxy open acid forms and pharmaceutically acceptable salts and esters thereof, including but not limited to lovastatin (see U.S. Pat. No. 4,342,767), simvastatin (see U.S. Pat. No. 4,444,784), dihydroxy open-acid simvastatin, particularly the ammonium or calcium salts thereof, pravastatin, particularly the sodium salt thereof (see U.S. Pat. No. 4,346,227), fluvastatin particularly the sodium salt thereof (see U.S. Pat. No. 5,354,772), atorvastatin, particularly the calcium salt thereof (see U.S. Pat. No. 5,273,995), cerivastatin, particularly the sodium salt thereof (see U.S. Pat. No. 5,177,080), pitavastatin also referred to as NK-104 (see PCT international publication number WO 97/23200) and ZD4522 (I will fill in more details here); HMG-CoA synthase inhibitors; squalene epoxidase inhibitors; squalene synthetase inhibitors (also known as squalene synthase inhibitors), acyl-coenzyrne A: cholesterol acyltransferase (ACAT) inhibitors including selective inhibitors of ACAT-1 or ACAT-2 as well as dual inhibitors of ACAT-1 and -2; microsomal triglyceride transfer protein (MTP) inhibitors; probucol; niacin; bile acid sequestrants; LDL (low density lipoprotein) receptor inducers; platelet aggregation inhibitors, for example glycoprotein IIb/IIIa fibrinogen receptor antagonists and aspirin; human peroxisome proliferator activated receptor gamma (PPARO) agonists including the compounds commonly referred to as glitazones for example troglitazone, pioglitazone and rosiglitazone and, including those compounds included within the structural class known as thiazolidinediones as well as those PPAR□ agonists outside the thiazolidinedione structural class; PPAR□ agonists such as clofibrate, fenofibrate including micronized fenofibrate, and gemfibrozil; PPAR dual □/□ agonists;,vitamin B₆ (also known as pyridoxine) and the pharmaceutically acceptable salts thereof such as the HCl salt; vitamin B₁₂ (also known as cyanocobalamin); folic acid or a pharmaceutically acceptable salt or ester thereof such as the sodium salt and the methylglucaamine salt; anti-oxidant vitamins such as vitamin C and E and beta carotene; beta-blockers; angiotensin II antagonists such as losartan; angiotensin converting enzyme inhibitors such as enalapril and captopril; calcium channel blockers such as nifedipine and diltiazam; endothelian antagonists; agents that enhance ABCA1 gene expression; FXR ligands including both inhibitors and agonists; bisphosphonate compounds such as alendronate sodium; and cyclooxygenase-2 inhibitors such as rofecoxib and celecoxib. Additionally, the compounds of Formula I of this invention, may be used in combination with anti-retroviral therapy in AIDS infected patients to treat lipid abnormalities associated with such treatment, for example but not limited to their use in combination with HIV protease inhibitors such as indinavir, nelfinavir, ritonavir and saquinavir.

Still another type of agent that can be used in combination with the compounds of this invention are cholesterol absorption inhibitors. Cholesterol absorption inhibitors block the movement of cholesterol from the intestinal lumen into enterocytes of the small intestinal wall. This blockade is their primary mode of action in reducing serum cholesterol levels. These compounds are distinct from compounds which reduce serum cholesterol levels primarily by mechanisms of action such as acyl coenzyme A—cholesterol acyl transferase (ACAT) inhibition, inhibition of triglyceride synthesis, MTP inhibition, bile acid sequestration, and transcription modulation such as agonists or antagonists of nuclear hormones. Cholesterol absorption inhibitors are described in U.S. Pat. No. 5,846,966, U.S. Pat. No. 5,631,365, U.S. Pat. No. 5,767,115, U.S. Pat. No. 6,133,001, U.S. Pat. No. 5,886,171, U.S. Pat. No. 5,856,473, U.S. Pat. No. 5,756,470, U.S. Pat. No. 5,739,321, U.S. Pat. No. 5,919,672, WO 00/63703, WO/0060107, WO 00/38725, WO 00/34240, WO 00/20623, WO 97/45406, WO 97/16424, WO 97/16455, and WO 95/08532, the entire contents of all of which are hereby incorporated by reference.

An exemplary cholesterol absorption inhibitor is ezetimibe, also known as SCH-58235, which is 1-(4-fluorophenyl)-3(R)-[3(S)-(4-fluorophenyl)-3-hydroxypropyl)]-4(S)-(4-hydroxyphenyl)-2-azetidinone, described in U.S. Pat. Nos. 5,767,115 and 5,846,966 and shown below as

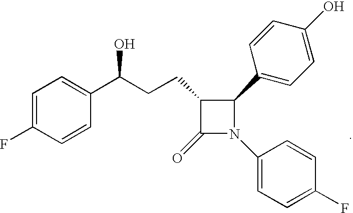

Additional exemplary hydroxy-substituted azetidinone cholesterol absorption inhibitors are specifically described in U.S. Pat. No. 5,767,115, column 39, lines 54-61 and column 40, lines 1-51 (hereby incorporated by reference), represented by the formula 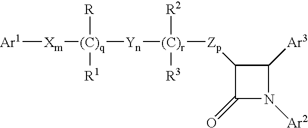
as defined in column 2, lines 20-63 (hereby incorporated by reference). These and other cholesterol absorption inhibitors can be identified according to the assay of hypolipidemic compounds using the hyperlipidemic hamster described in U.S. Pat. No. 5,767,115, column 19, lines 47-65 (hereby incorporated by reference), in which hamsters are fed a controlled cholesterol diet and dosed with test compounds for seven days. Plasma lipid analysis is conducted and data is reported as percent reduction of lipid versus control.

Therapeutically effective amounts of cholesterol absorption inhibitors include dosages of from about 0.1 to about 30 mg/kg of body weight per day, preferably about 0.1 to about 15 mg/kg. For an average body weight of 70 kg, the dosage level is therefore from about 7 mg to about 2100 mg of drug per day, e.g. 10, 20, 40, 100 and 200 mg per day, preferably given as a single daily dose or in divided doses two to six times a day, or in sustained release form. This dosage regimen may be adjusted to provide the optimal therapeutic response when the cholesterol absorption inhibitor is used in combination with a compound of the instant invention. A therapeutically or prophylactically effective amount, as appropriate, of a compound of Formula I can be used for the preparation of a medicament useful for treating lipid disorders, particularly for treating low HDL cholesterol levels as well as for treating and/or reducing the risk for diseases and conditions affected by agonism of LXR, preventing or reducing the risk of developing atherosclerotic disease, halting or slowing the progression of atherosclerotic disease once it has become clinically manifest, and preventing or reducing the risk of a first or subsequent occurrence of an atherosclerotic disease event. For example, the medicament may be comprised of about 0.5 mg to 7 g of a compound of Formula I, or more particularly about 0.5 mg to 3.5 g. The medicament comprised of a compound of Formula I may also be prepared with one or more additional active agents, such as those described supra.

As used herein, the term LXR includes all subtypes of this receptor. The compounds of Formula I are LXR ligands and individually may vary in their selectivity for one or the other of LXRα and LXRβ, or they may have mixed binding affinity for both LXRα and LXRβ. More particularly, the tested compounds included within the scope of this invention have an IC₅₀ less than or equal to 1 □M for at least one of either the LXRα or LXR□ receptors employing the LXR radioligand competition scintillation proximity assays described below in the Example section.

Compound A is used in the following assays and has the following structural formula:

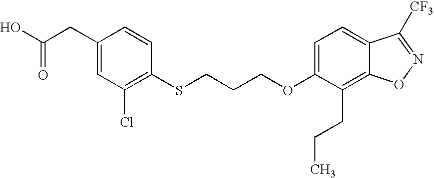

### Compound A

Compound A and related compounds are disclosed along with methods for making them in WO97/28137 herein incorporated by reference in its entirety (U.S. Ser. No. 08/791211, filed Jan. 31, 1997).

The compounds in the following examples were characterized using ¹H NMR at 400 or 500 MHz field strength, and/or by ESI mass spectroscopy (MS).

### EXAMPLE 1

Radioligand Competition Binding Scintillation Proximity Assays:

Preparation of Recombinant Human LXR□ and LXR□:

Human LXR□ and LXR□ were expressed as GST-fusion proteins in E. coli. The ligand binding domain cDNAs for human LXR□ (amino acids 164-447) and human LXR□ (amino acids 149-455) were subdloned into the pGEX-KT expression vector (Pharmacia). E. coli containing the respective plasmids were propagated, induced, and harvested by centrifugation. The resuspended pellet was broken in a French press and debris was removed by centrifugation. Recombinant human LXR receptors were purified by affinity chromatography on glutathione sepharose and receptor was eluted with glutathione. Glycerol was added to a final concentration of 50% to stabilize the receptor and aliquots were stored at −80° C.

Binding to LXRα:

For each assay, an aliquot of human GST-LXRα receptor was incubated in a final volume of 100 □l SPA buffer (10 mM Tris, pH 7.2, 1 mM EDTA, 10% glycerol, 10 mM Na molybdate, 1 mM dithiothreitol, and 2 μg/ml benzamidine) containing 1.25 mg/ml yttrium silicate protein A coated SPA beads (Amersham Pharmacia Biotech, Inc.), 8.3 □g/ml anti-GST antibody (Amersham Pharmacia Biotech, Inc.), 0.1% non-fat dry milk and 25 nM [³H₂] Compound A (13.4 Ci/mmole), ±test compound. After incubation for ˜16 h at 15° C. with shaking, the assay plates were counted in a Packard Topcount. In this assay the K_{d} for Compound A for LXR□ is ≈15 nM.

Binding to LXRβ:

For each assay, an aliquot of human GST-LXRβ ligand binding domain receptor was incubated in a final volume of 100 □l SPA buffer (10 mM Tris, pH 7.2, 1 mM EDTA, 10% glycerol, 10 mM Na molybdate, 1 mM dithiothreitol, and 2 μg/ml benzamidine) containing 1.25 mg/ml yttrium silicate protein A coated SPA beads (Amersham Pharmacia Biotech, Inc.), 8.3 □g/ml anti-GST antibody (Amersham Pharmacia Biotech, Inc.) 0.1% non-fat dry milk and 25 nM [³H₂]Compound A (13.4 Ci/mmole), ± test compound. After incubation for ˜16 h at 15° C. with shaking, the assay plates were counted in a Packard Topcount. In this assay the K_{d} for Compound A for LXR□ is ≈10 nM.

Results:

Representative tested compounds of Formula I are ligands for human LXR□ and human LXR□ each having an IC₅₀ less than or equal to 900 nM for the LXR□ receptor, and an IC₅₀ less than or equal to 5,000 nM for the LXR□ receptor.

### EXAMPLE 2

Transactivation Assay

Plasmids

Expression constructs were prepared by inserting the ligand binding domain (LBD) of human LXR□ and LXR□ cDNAs adjacent to the yeast GAL4 transcription factor DNA binding domain (DBD) in the mammalian expression vector pcDNA3 to create pcDNA3-LXRα/GAL4 and pcDNA3-LXR□/GAL4, respectively. The GAL4-responsive reporter construct, pUAS(5X)-tk-luc, contained 5 copies of the GAL4 response element placed adjacent to the thymidine kinase minimal promoter and the luciferase reporter gene. The transfection control vector, pEGFP-N1, contained the Green Fluorescence Protein (GFP) gene under the regulation of the cytomegalovirus promoter.

Assay

HEK-293 cells were seeded at 40,000 cells/well in 96 well plates in Dulbecco's modified Eagle medium (high glucose) containing 10% charcoal stripped fetal calf serum, 100 units/ml Penicillin G and 100 μg/ml Streptomycin sulfate at 37° C. in a humidified atmosphere of 5% CO₂. After 24 h, transfections were performed with Lipofectamine (Gibco-BRL, Gaithersburg, Md.) according to the instructions of the manufacturer. In general, transfection mixes contained 0.002 □g of LXR□/GAL4 or LXR□/GAL4 chimeric expression vectors, 0.02 □g of reporter vector pUAS(5X)-tk-luc and 0.034 □g of pEGFP-N1 vector as an internal control of transfection efficiency. Compounds were characterized by incubation with transfected cells for 48 h across a range of concentrations. Cell lysates were prepared from washed cells using Cell Lysis Buffer (Promega) according to the manufacturer's directions. Luciferase activity in cell extracts was determined using Luciferase Assay Buffer (Promega) in a ML3000 luminometer (Dynatech Laboratories). GFP expression was determined using the Tecan Spectrofluor Plus at excitation wavelength of 485 nm and emission at 535 nm. Luciferase activity was normalized to GFP expression to account for any variation in efficiency of transfection.

Results with representative tested compounds of Formula I for LXR□ transactivation are EC₅₀3 to 3,000 nM, and results for LXR□ transactivation are EC₅₀ of 3 to 3,000 nM.

### EXAMPLE 3

### Step 1 Preparation of 2,4-dihydroxy-3-propyl-1′,1′,1′-trifluoroacetophenone

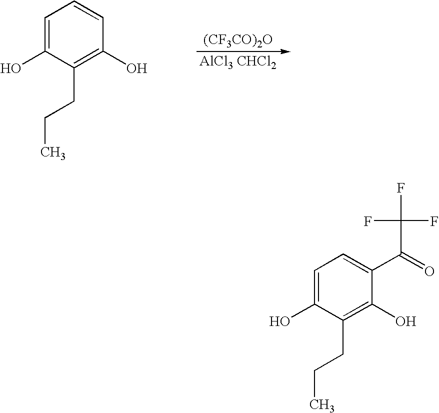

A solution of 2-propylresorcinol (5.0 grams) and trifluoroacetic anhydride (9.6 mL) in 1,2-dichloroethane (30.0 mL) was treated with aluminum chloride(4.38 grams). This mixture was stirred overnight. The reaction mixture was partitioned between methylene chloride and water. The organic phase was dried over sodium sulfate and filtered. The solvent was evaporated and the resulting solid was recrystallized from methylene chloride and cyclohexane (1:1) to give the titled compound.

¹H NMR (CDCl₃) □ 7.59 (d, 1H), 6.24 (d, 1H), 5.92 (s, 1H), 2.63 (t, 2H), 1.74 (s, 1H), 1.58 (m, 2H), 0.98 (t, 3H).

### Step 2 Preparation of 3-trifluoromethyl-7-propyl-6-hydroxybenzisoxazole

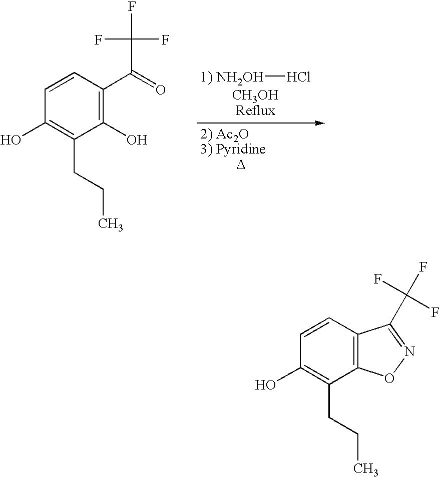

A mixture of 2,4-dihydroxy-3-propyl-1′,1′,1′-trifluoroacetophenone(2.5 grams), sodium acetate (4.18 grams), hydroxylamine hydrochloride (3.59 grams) and methanol (80 mL) was heated under reflux overnight. The solvent was then evaporated and the resulting solid was partitioned between ethyl acetate and pH 7 buffer. The organic phase was separated and washed with brine. The organic phase was dried over sodium sulfate and the solvent was evaporated to give a oil. The oil was then dissolved in acetic anhydride. The solution was stirred for two hours, then the acetic anhydride was evaporated in vac. The residue was partitioned between ethyl acetate and pH 7 buffer and the organic phase was dried over sodium sulfate. The organic phase was evaporated to give an oil. This was dissolved in pyridine and refluxed overnight. The solvent was evaporated in vac to give an oil which was chromatographed on silica gel using ethyl acetate and hexane (1:4) to give the titled compound.

¹H NMR (CDCl₃) □ 7.46 (d, 1H), 6.92 (d, 1H), 5.42 (bs, 1H), 2.89 (t, 2H), 1.74 (m, 2H), 0.98 (t, 3H).

### EXAMPLE 4

### Preparation of 6-Hydroxy-3-neopentyl-7-propyl-1,2-benzisoxazole

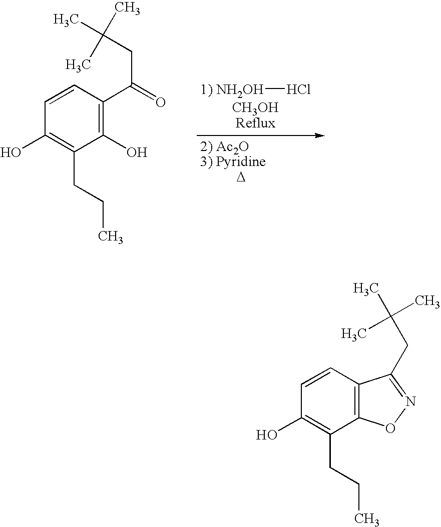

1-(2,4-dihydroxy-3-propylphenyl)-3,3-dimethylbutan-1-one (200 gm, 0.8 mole), prepared as in Example 4 Step 1, was converted to 6-Hydroxy-3-neopentyl-7-propyl-1,2-benzisoxazole as for Example 1 Step 2 above using hydroxylamine hydrochloride (278 gm, 4 mole) and sodium acetate (320 gm) in methanol (2.5 L). A second addition of hydroxylamine hydrochloride (106 gm, 1.5 mole) and sodium acetate (250 gm) was made after 18 Hr at reflux followed by further heating under reflux for a total of 36 hrs. After isolation of the oxime as above the crude material was purified by crystallization from hexanes. Conversion to the oxime acetate was accomplished as described in Example 4 Step 2. Full conversion requires 18 hrs for this case. Ring closure in pyridine as for Example 1 Step 2 yields a dark oil. The crude product was eluted from SiO₂ (300 gm) with CH₂Cl₂. The resulting oil was crystallized from hexanes:ether to yield the desired 6-hydroxy-3-neopentyl-7-propyl-1,2-benzisoxazole.

¹HNMR (CDCl₃) □ 7.33 (d, 1H, J=8.5 Hz), 6.81 (d, 1H, J=8.5 Hz), 5.07 (brd, 1H), 289 (collapsed dd, 2H), 177 (sect, 2H, J=7.5 Hz), 1.08 (s, 9H), 1.04 (t, 3H, J=7.3 Hz).

### EXAMPLE 5

### Step 1 Preparation of ethyl 4-{[7-propyl-3-(trifluoromethyl)-1,2-benzisoxazol-6-yl]oxy}butyrate

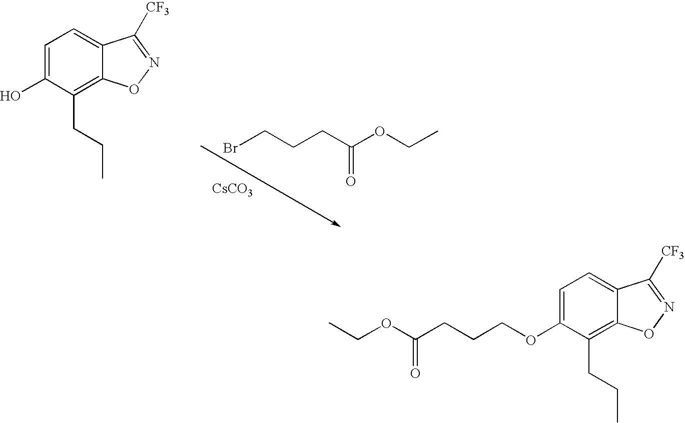

To a DMF solution (30 mL) of 6-hydroxy-7-propyl-3-(trifluoromethyl)-1,2-benzisoxazole from Example 3 Step 2 (1.5 g, 6.12 mmol) was added ethyl 3-bromopropionate (1.05 mL, 7.35 mmol), followed by CsCO₃ (2.13 g, 6.55 mmol). The mixture was stirred at room temperature overnight. After aqueous work-up (ether) and chromatography on silica gel using ethyl acetate and hexane (1:9) the titled compound was obtained.

¹H NMR (CDCl₃) □ 7.54 (d, 1H, J=8.5 Hz), 7.04 (d, 1H), 4.14 (m, 4H), 2.89 (t, 2H, J=7.0), 2.55 (t, 2H, J=7.0), 2.17 (m, 2H), 1.70(m, 2H), 1.26 (t, 3H, J=7.0), 0.96 (t, 3H, J=7.5).

MS: m/z=360 (M+H)

### Step 2 Preparation of 4-{[7-propyl-3-(trifluoromethyl)-1,2-benzisoxazol-6-yl]oxy}butyric acid

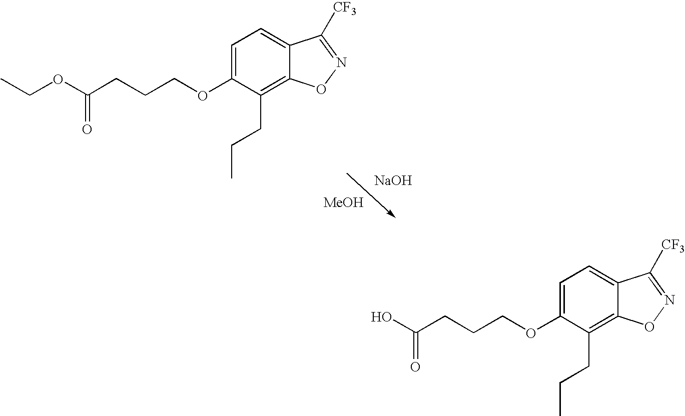

To a CH₃OH solution (100 mL) of ethyl 4-{[7-propyl-3-(trifluoromethyl)-1,2-benzisoxazol-6-yl]oxy}butyrate (6.59 g, 25.5 mmol) was added NaOH (1 N, 73.4 mL, 73.4 mmol). The mixture was stirred at room temperature overnight. After aqueous work-up (ethyl acetate) and chromatography on silica gel using ethyl acetate, hexane and acetic acid (30:70:2.5) the titled compound was obtained.

¹H NMR (CDCl₃) □ 7.55 (d, 1H, J=8.5 Hz), 7.05 (d, 1H, J=9.0), 4.17 (t, 2H, J=6.0), 2.91 (t, 2H, J=7.0), 2.64 (t, 2H, J=7.5), 2.21 (m, 2H), 1.71(m, 2H), 0.97 (t, 3H, J=7.5).

MS: m/z=332 (M+H)

### Step 3 Preparation of N,N-dimethyl-4-{[7-propyl-3-(trifluoromethyl)-1,2-benzisoxazol-6-yl]oxy}butyramide

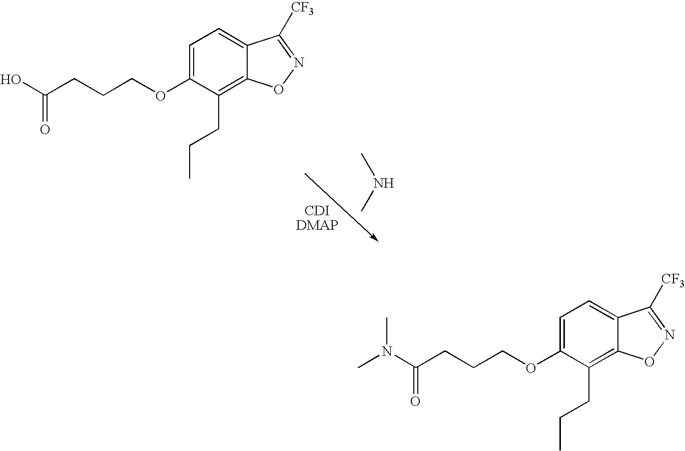

To a CH₂Cl₂ solution (2 mL) of 4-{[7-propyl-3-(trifluoromethyl)-1,2-benzisoxazol-6-yl]oxy}butyric acid (50 mg, 0.15 mmol) was added CDI (30 mg, 0.18 mmol) and DMAP (catalyst). The mixture was stirred at room temperature for 1 hour, followed by addition of N,N dimethyl amine (0.15 uL, 0.30 mmol), then further stirring at room temperature overnight. The solvent was evaporated and the material was purified by chromatography on silica gel using ethyl acetate and hexane (8:2) to give the titled compound.

¹H NMR (CDCl₃) □ 7.55 (d, 1H, J=8.5 Hz), 7.08 (d, 1H, J=9.0), 4.19 (t, 2H, J=6.0), 3.03 (s, 3H), 2.97 (s, 3H), 2.91 (t, 2H, J=7.5), 2.56 (t, 2H, J=7.0), 2.21 (m, 2H), 1.71(m, 2H), 0.97 (t, 3H, J=7.5).

MS: m/z=359 (M+H)

### EXAMPLE 6

### Preparation N-methyl-4-{[7-propyl-3-(trifluoromethyl)-1,2-benzisoxazol-6-yl]oxy}butyramide

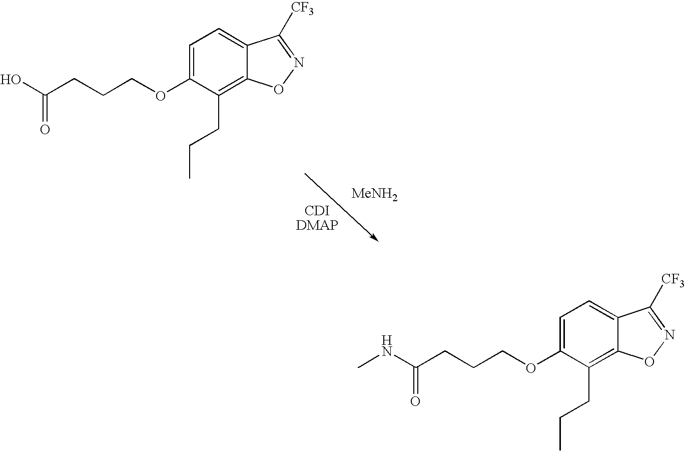

To a CH₂Cl₂ solution (2 mL) of 4-{[7-propyl-3-(trifluoromethyl)-1,2-benzisoxazol-6-yl]oxy}butyric acid from Example 5 Step 2 (40 mg, 0.12 mmol) was added CDI (23.5 mg, 0.145 mmol) and DMAP (catalyst). The mixture was stirred at room temperature for 1 hour, followed by addition of Methyl amine (0.12 uL, 0.24 mmol), then further stirring at room temperature overnight. The solvent was evaporated and the material was purified by chromatography on silica gel using ethyl acetate and hexane (7:3) to give the titled compound.

¹H NMR (CDCl₃) □ 7.55 (d, 1H, J=8.5 Hz), 7.06 (d, 1H, J=9.0), 5.44(sb, 1H), 4.15 (t, 2H, J=6.5), 2.91 (t, 2H, J=7.0), 2.83 (d, 3H, J=4.5), 2.41 (t, 2H, J=7.5), 2.22 (m, 2H), 1.71 (m, 2H), 0.97 (t, 3H, 7.5 Hz).

MS: m/z=345 (M+H)

### EXAMPLE 7

### Step 1 Preparation of 7-propyl-3-neopentyl-6-(3-bromopropyloxy)-1,2-benzisoxazole

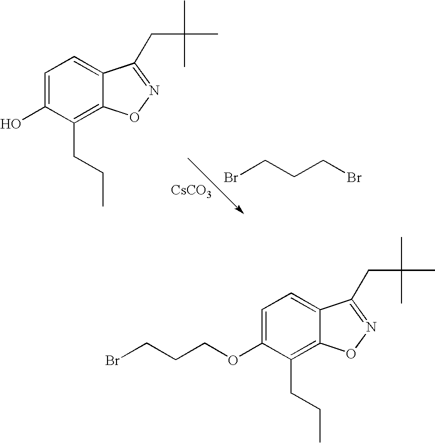

To a DMF solution (30 mL) of 6-hydroxy-7-propyl-3neopentyl-1,2-benzisoxazole from Example 4 (2.0 g, 8.0 mmol) was added CsCO₃ (2.83 g, 8.67 mmol), followed by 1,3-dibromopropane (2.47 mL, 24.3 mmol) the mixture was stirred at room temperature for 16 hours. After aqueous work-up (ether) and chromatography on silica gel using ethyl acetate and hexane (1:19) the titled compound was obtained.

¹H NMR (CDCl₃) □ 77.37 (d, 1H, J=8.8 Hz), 6.92 (d, 1H, J=8.6 Hz), 4.20 (t, 2H, J=5.7 Hz), 3.65 (t, 2H, J=6.4 Hz), 2.87 (m, 2H), 2.37 (pent, 2H, J=6.3 Hz), 1.71 (sext, 2H, J=7.5 Hz), 1.05 (s, 1H), 0.97 (t, 3H, J=7.4 Hz).

MS: m/z=369 (M+H)

### Step 2 Preparation of 7-propyl-3-neopentyl-6-(3-cyanopropyloxy)-1,2-benzisoxazole

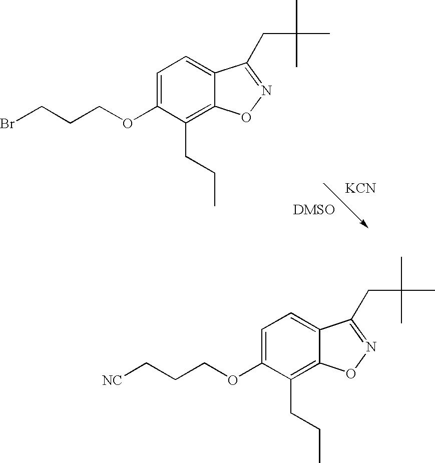

To a DMSO solution (200 mL) of 7-propyl-3-neopentyl-6-(3-bromopropyloxy)-1,2-benzisoxazole (2.27 g, 6.18 mmol) was added KCN (0.81 g, 12.4 mmol). The mixture was stirred at 60° C. for 3 hours. After aqueous work-up (ethyl acetate) and chromatography on silica gel using ethyl acetate and hexane (1:4), the titled compound was obtained.

MS: m/z=315 (M+H)

### Step 3 Preparation of 4-{[7-propyl-3-neopentyl-1,2-benzisoxazol-6-yl]oxy}butyric acid

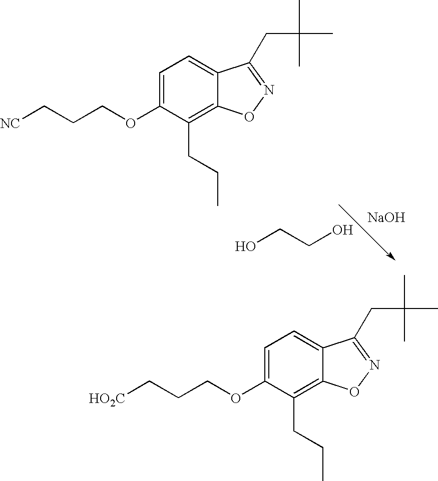

To an ethylene glycol solution (30 mL) of 7-propyl-3-neopentyl-6-(3-cyanopropyloxy)-1,2-benzisoxazole (1.25 g, 4.0 mmol) was added NaOH (16.0 mL, 32.0 mmol). The mixture was heated at 100° C. overnight. After neutralized by 1N HCl , followed by aqueous work-up (ether) and chromatography on silica gel using methanol and dichloromethane (1:9), the titled compound was obtained.

¹H NMR (CDCl₃) □ 7.36 (d, 1H, J=8.7 Hz), 6.89 (d, 1H, J=8.7 Hz), 4.12 (t, 2H, J=6.0 Hz), 2.87 (t, 2H, J=7.6 Hz), 2.81 (s, 2H), 2.63 (t, 2H, J=7.2 Hz), 2.18 (pent, 2H, J=6.6 Hz), 1.70 (sext, 2H, J=7.4 Hz), 1.05 (s, 9H), 0.971 (t, 3H, J=7.4 Hz).

MS: m/z=334 (M+H)

### Step 4 Preparation of N,N-Dimethyl 4-{[7-propyl-3-neopentyl-1,2-benzisoxazol-6-yl]oxy}butyramide

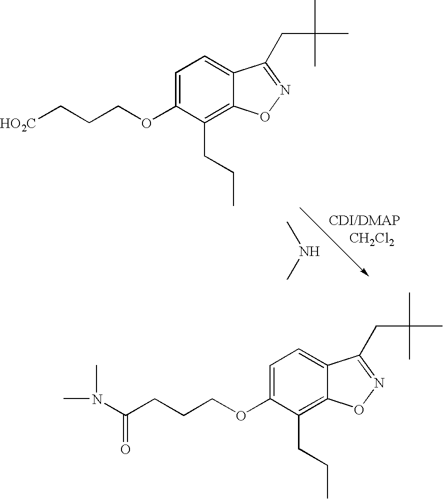

To a methylene chloride solution (2.0 mL) of the acid from step3 (19.0 mg, 0.057 mmol) was added CDI (11.1 mg, 0.069 mmol) and DMAP (catalyst), then stirred at room temperature for 1 hour. N,N-dimethyl amine (0.28 mL, 0.57 mmol) was added and the mixture was stirred at room temperature overnight. After aqueous work-up (ethyl acetate) and chromatography on silica gel using ethyl acetate and hexane (9:1), the titled compound was obtained.

¹H NMR (CDCl₃) □ 7.26 (d, 1H, J=8.7 Hz), 6.82 (d, 1H, J=8.7 Hz), 4.04 (t, 2H, J=5.9 Hz),2.93 (s, 3H), 2.87 (s, 3H), 2.78 (t, 2H, J=7.6 Hz), 2.71 (s, 2H), 2.48 (t, 2H, J=7.2 Hz), 2.10 (pent, 2H, J=6.6 Hz), 1.62 (sext, 2H, J=7.4 Hz), 0.95 (s, 9H), 0.877 (t, 3H, J=7.4 Hz).

MS: m/z=361 (M+H)

### EXAMPLE 8

### Preparation of N-Methyl 4-{[7-propyl-3-neopentyl-1,2-benzisoxazol-6-yl]oxy}butyramide

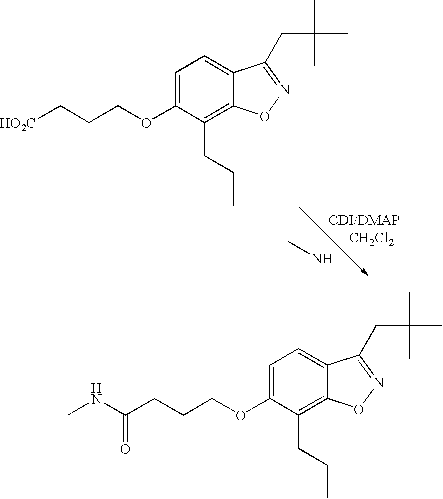

To a methylene chloride solution (2.0 mL) of the acid from Example 7 Step 3 (19.0 mg, 0.057 mmol) was added CDI (11.1 mg, 0.069 mmol) and DMAP (catalyst), then stirred at room temperature for 1 hour. N-methyl amine (0.28 mL, 0.57 mmol) was added and the mixture was stirred at room temperature overnight. After aqueous work-up (ethyl acetate) and chromatography on silica gel using ethyl acetate and hexane (9:1), the titled compound was obtained.

¹H NMR (CDCl₃) □ 7.28 (d, 1H, J=8.7 Hz), 6.82 (d, 1H, J=8.7 Hz), 4.01 (t, 2H, J=6.0 Hz), 2.72-2.86 7H overlapping, 2.35 (t, 2H, J=7.2 Hz), 2.10 (pent, 2H, J=6.6 Hz), 1.60 (sext, 2H, J=7.4 Hz), 0.957 (s, 9H), 0.872 (t, 3H, J=7.4 Hz).

MS: m/z=347 (M+H)

### EXAMPLE 9

### Preparation of N-Ethyl 4-{[7-propyl-3-neopentyl-1,2-benzisoxazol-6-yl]oxy}butyramide

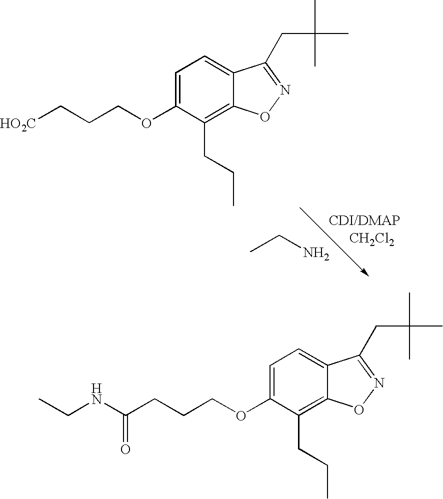

To a methylene chloride solution (2.0 mL) of the acid from Example 7 Step 3 (46.8 mg, 0.141 mmol) was added CDI (27.4 mg, 0.17 mol) and DMAP (catalyst), then stirred at room temperature for 1 hour. N-ethyl amine (0.7 mL, 1.4 ol) was added and the mixture was stirred at room temperature overnight. After aqueous work-up (ethyl acetate) and chromatography on silica gel using ethyl acetate and hexane (7:3) the titled compound was obtained.

¹H NMR (CDCl₃) □ 7.35 (d, 1H, J=8.7 Hz), 6.89 (d, 1H, J=8.7 Hz), 4.10 (t, 2H, J=5.9 Hz), 3.30 (m, 2H), 2.87 (t, 2H, J=7.6 Hz), 2.81 (s, 2H), 2.40 (t, 2H, J=7.2 Hz), 2.18 (pent, 2H, J=6.6 Hz), 1.70 (sext, 2H, J=7.4 Hz), 1.12 (t, 3H, J=7.2 Hz, 1.04 (s, 9H), 0.966 (t, 3H, J=7.4 Hz).

MS: m/z=361(M+H)

### EXAMPLE 10

### Preparation of N,N-Diethyl 4-{[7-propyl-3-neopentyl-1,2-benzisoxazol-6-yl]oxy}butyramide

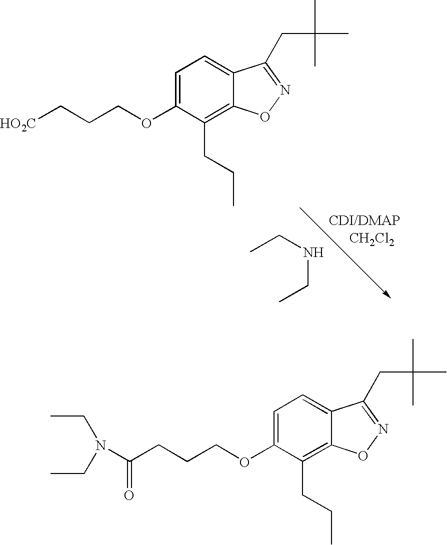

To a methylene chloride solution (2.0 mL) of the acid from Example 7 Step 3 (35.8 mg, 0.11 mol) was added CDI (21.0 mg, 0.13 mmol) and DMAP (catalyst), then stirred at room temperature for 1 hour. N,N-diethyl amine (0.5 mL, 1.1 mmol) was added and the mixture was stirred at room temperature overnight. After aqueous work-up (ethyl acetate) and chromatography on silica gel using ethyl acetate and hexane (7:3) the titled compound was obtained.

¹H NMR (CDCl₃) □ 7.36 (d, 1H, J=8.7 Hz), 6.91 (d, 1H, J=8.7 Hz), 4.13 (t, 2H, J=6.9 Hz), 3.39 (q, 2H, J=7.1 Hz), 3.32 (q, 2H, J=7.1 Hz), 2.87 (d, 2H, J=7.7 Hz), 2.80 (s, 2H), 2.55 (t, 2H, J=7.3 Hz), 2.19 (pent, 2H, J=6.7 Hz), 1.70 (sext, 2H, J=7.4 Hz), 1.17 (t, 3H, J=7.2 Hz), 1.12 (t, 3H, J=7.1 Hz), 1.05 (s, 9H), 0.966 (t, 3H, J=7.4 Hz).

MS: m/z=389(M+H)

### EXAMPLE 11

### Preparation of 4-{[7-propyl-3-neopentyl-1,2-benzisoxazol-6-yl]oxy}butyrlpiperidine

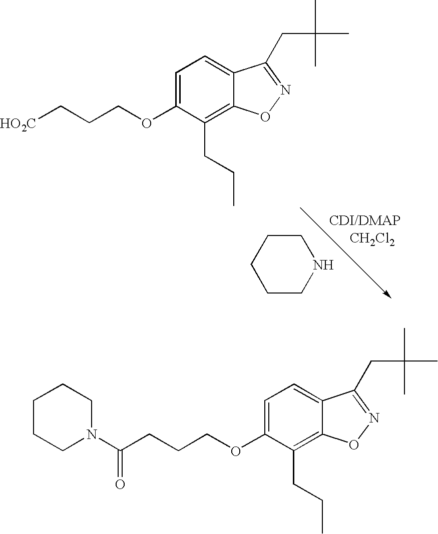

To a methylene chloride solution (2.0 mL) of the acid from Example 7 Step 3 (42.9 mg, 0.13 mmol) was added CDI (25.1 mg, 0.16 mmol) and DMAP (catalyst), then stirred at room temperature for 1 hour. Piperidine (0.13 mL, 1.29 mmol) was added and the mixture was stirred at room temperature overnight. After aqueous work-up (ethyl acetate) and chromatography on silica gel using ethyl acetate and hexane (1:1) the titled compound was obtained.

¹H NMR (CDCl₃) □ 7.27 (d, 1H, J=8.7 Hz), 6.82 (d, 1H, J=8.7 Hz), 4.04 (t, 2H, J=5.9 Hz), 3.47 (m, 2H), 3.33 (m, 2H), 2.78 (t, 2H, J=7.5 Hz), 2.71 (s, 2H), 2.47 (t, 2H, J=7.2 Hz), 2.08 (pent, 2H, J=6.6 Hz), 1.61 (sext, 2H, J=7.4 Hz), 1.55 (m, 2H), 1.46 (m, 4H), 0.96 (s, 9H), 0.883 (t, 3H, J=7.3 Hz).

MS: m/z=401 (M+H)

### EXAMPLE 12

### Preparation of N-Propyl 4-{[7-propyl-3-neopentyl-1,2-benzisoxazol-6-yl]oxy}butyramide

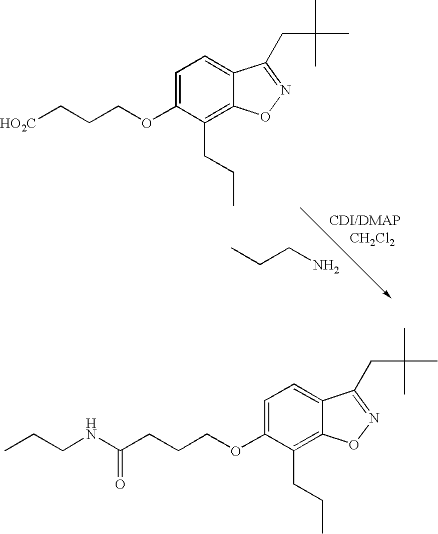

To a methylene chloride solution (2.0 mL) of the acid from Example 7 Step 3 (36.8 mg, 0.11 mmol) was added CDI (22.0 mg, 0.13 mmol) and DMAP (catalyst), then stirred at room temperature for 1 hour. N, propyl amine (91 uL, 1.1 mmol) was added and the mixture was stirred at room temperature overnight. After aqueous work-up (ethyl acetate) and chromatography on silica gel using ethyl acetate and hexane (6:4) the titled compound was obtained.

¹H NMR (CDCl₃) □ 7.38 (d, 1H, J=8.5 Hz), 6.92 (d, 1H, J=8.5), 5.46 (bs, 1H), 4.13 (t, 2H, J=6.0), 3.25 (q, 2H, J=7.0 and 13.5), 2.90 (t, 2H, J=7.5), 2.83 (s, 2H), 2.43 (t, 2H, J=7.0), 2.21 (m, 2H), 1.74 (m, 2H), 1.59 (m, 2H), 1.18 (t, 3H, J=7.5), 1.04 (s, 9H), 0.97 (t, 3H, J=7.5).

MS: m/z=375(M+H)

### EXAMPLE 13

### Preparation of N-(2-Furyl)methyl 4-{[7-propyl-3-neopentyl-1,2-benzisoxazol-6-yl]oxy}butyramide

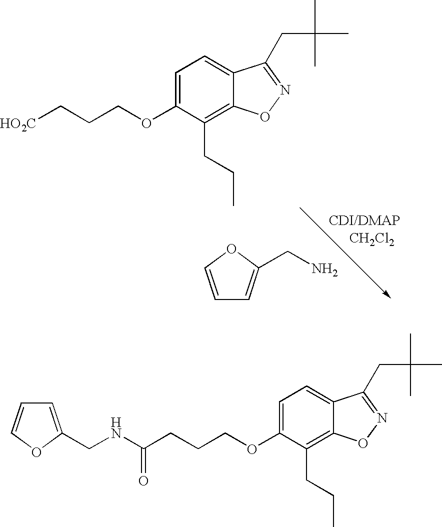

To a methylene chloride solution (2.0 mL) of the acid from Example 7 Step 3 (41.7 mg, 0.13 mmol) was added CDI (41.0 mg, 0.25 mmol) and DMAP (catalyst), then stirred at room temperature for 1 hour. Furfuryl amine (0.11 mL, 1.25 mmol) was added and the mixture was stirred at room temperature overnight. After aqueous work-up (ethyl acetate) and chromatography on silica gel using ethyl acetate and hexane (1:1) the titled compound was obtained.

¹H NMR (CDCl₃) □ 7.27 (d, 1H, J=8.5 Hz), 7.25 (d, 1H, J=), 6.21 (t, 1H, J=3.0), 6.12 (d, 1H, J=3.0), 5.81 (bs, 1H), 4.36 (d, 2H, J=5.5), 4.02 (t, 2H, J=5.5), 2.77 (t, 2H, J=7.5), 2.72 (s, 2H), 2.37 (t, 2H, 7.5), 2.10 (m, 2H), 1.62 (m, 2H), 0.97 (s, 9H), 0.88 (t, 3H, J=7.0).

MS: m/z=413.5(M+H)

### EXAMPLE 14

### Preparation of N-Butyl 4-{[7-propyl-3-neopentyl-1,2-benzisoxazol-6-yl]oxy}butyramide

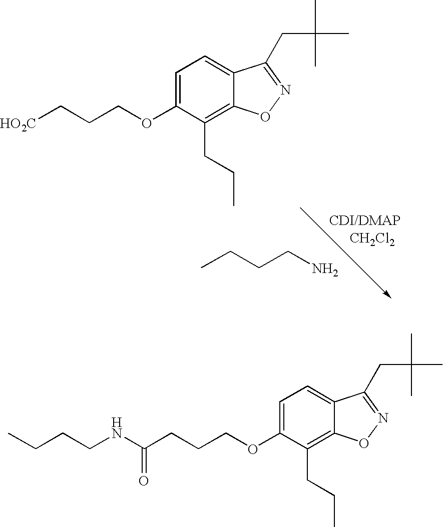

To a methylene chloride solution (2.0 mL) of the acid from Example 7 Step 3 (60.0 mg, 0.18 mmol) was added CDI (35.1 mg, 0.22 mmol) and DMAP (catalyst), then stirred at room temperature for 1 hour. Butyl amine (0.18 mL, 1.8 mmol) was added and the mixture was stirred at room temperature overnight. After aqueous work-up (ethyl acetate) and chromatography on silica gel using ethyl acetate and hexane (6:4) the titled compound was obtained.

¹H NMR (CDCl₃) □ 7.35 (d, 1H, J=8.7 Hz), 6.89 (d, 1H, J=8.7 Hz), 4.10 (t, 2H, J=6.0 Hz), 3.25 (q, 2H, J=6.5 Hz), 2.87 (t, 2H, J=7.6 Hz), 2.80 (s, 2H), 2.40 (t, 2H, J=7.2 Hz), 2.18 (pent, 2H, J=6.5 Hz), 1.70 (sext, 2H, J=7.4 Hz), 1.45 (m, 2H), 1.32 (m, 2H), 1.05 (s, 9H), 0.971 (t, 3H, J=7.4 Hz), 0.891 (t, 3H, J=7.3 Hz).

MS: m/z=389(M+H)

### EXAMPLE 15

### Preparation of 4-{[7-propyl-3-(trifluromethyl)-1,2-benzisoxazol-6-yl]oxy}butyramide

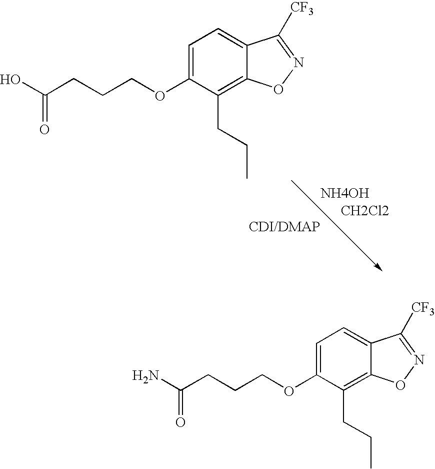

To a CH₂Cl₂ solution (2.0 mL ) of 4-{[7-propyl-3-(trifluoromethyl)-1,2-benzisoxazol-6-yl]oxy}butyric acid from Example 5 Step 2 (53 mg, 0.16mmol) was added CDI (30.8 mg, 0.19 mmol), DMAP (catalyst), then stirred at room temperature for 3 hours, followed by adding ammonium hydroxide (55.6 mg, 1.6 mmol). The mixture was stirred at room temperature overnight. After aqueous work-up (ethyl acetate) and chromatography on silica gel using methanol and methylene chloride (1:19) the titled compound was obtained.

¹H NMR (CDCl₃) □ 7.55 (d, 1H, J=8.9 Hz), 7.06 (d, 1H, J=8.7 Hz), 5.38 (brd, 1H), 5.33 (brd, 1H), 4.13 (t, 2H, J=6.1 Hz), 2.91 (t, 2H, J=7.4 Hz), 2.48 (t, 2H, J=7.2 Hz), 2.21 (pent, 2H, J=6.6 Hz), 1.70 (sext, 2H, J=7.5 Hz), 0.971 (t, 3H, J=7.4 Hz).

MS: m/z=331 (M+H)

### EXAMPLE 16

### Preparation of N-Propyl 4-{[7-propyl-3-(trifluromethyl)-1.2-benzisoxazol-6-yl]oxy}butyramide

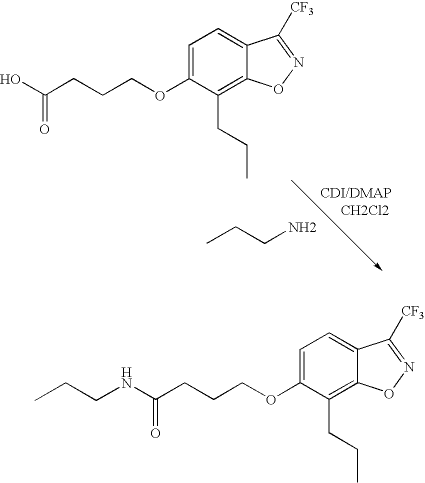

To a CH₂Cl₂ solution (2.0 mL) of 4-{[7-propyl-3-(trifluoromethyl)-1,2-benzisoxazol-6-yl]oxy}butyric acid from Example 5 Step 2 (50.0 mg, 0.15 mmol) was added CDI (30.0 mg, 0.18 mmol), DMAP (catalyst), then stirred at room temperature for 3 hours, followed by adding propyl amine (0.124 mL, 1.5 mmol). The mixture was stirred at room temperature overnight. After aqueous work-up (ethyl acetate) and chromatography on silica gel using ethyl acetate and hexane (8:2) the titled compound was obtained.

¹H NMR (CDCl₃) □ 7.55 (d, 1H, J=8.7 Hz), 7.06 (d, 1H, J=8.7 Hz), 4.16 (t, 2H, J=6.1 Hz), 3.23 (q, 2H, J=6.6 Hz), 2.91 (t, 2H, J=7.4 Hz), 2.40 (t, 2H, J=7.2 Hz), 2.20 (pent, 2H, J=6.6 Hz), 1.70 (sext, 2H, J=7.5 Hz), 1.51 (sext, 2H, J+7.3 Hz), 0.911 (t, 3H, J=7.4 Hz).

MS: m/z=373 (M+H)

### EXAMPLE 17

### Preparation of 4-{[7-propyl-3-(trifluromethyl)-1,2-benzisoxazol-6-yl]oxy}butyrylpiperidine

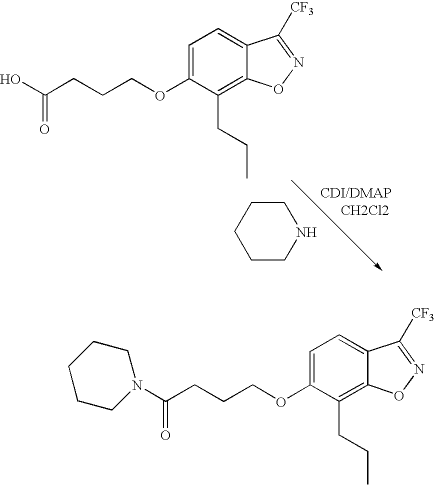

To a CH₂Cl₂ solution (2.0 mL) of 4-{[7-propyl-3-(trifluoromethyl)-1,2-benzisoxazol-6-yl]oxy}butyric acid from Example 5 Step 2 (44.0 mg, 0.13 mmol) was added CDI (25.9 mg, 0.16 mmol), DMAP (catalyst), then stirred at room temperature for 3 hours, followed by adding piperidine (0.13 mL, 1.3 mmol). The mixture was stirred at room temperature overnight. After aqueous work-up (ethyl acetate) and chromatography on silica gel using ethyl acetate and hexane (1:1) the titled compound was obtained.

¹H NMR (CDCl₃) □ 7.55 (d, 1H, J=8.7 Hz), 7.07 (d, 1H, J=8.7 Hz), 4.17 (t, 2H, J=6.0 Hz), 3.55 (m, 2H), 3.41 (m, 2H), 2.89 (t, 2H, J=7.5 Hz), 2.55 (t, 2H, J=7.2 Hz), 2.18 (pent, 2H, J=6.6 Hz), 1.70 (sext, 2H, J=7.4 Hz), 1.63 (m, 2H), 1.55 (m, 4H), 0.95 (t, 3H, J=7.4 Hz).

MS: m/z=399 (M+H)

### EXAMPLE 18

### Preparation of N-(4-carbomethoxyphenyl)methyl 4-{[7-propyl-3-(trifluromethyl)-1,2-benzisoxazol-6-yl]oxy}butyramide

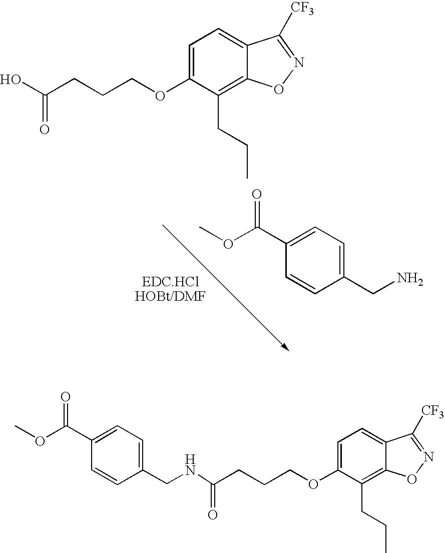

To a DMF solution (1.0 mL) of 4-{[7-propyl-3-(trifluoromethyl)-1,2-benzisoxazol-6-yl]oxy}butyric acid from Example 5 Step 2 (80.0 mg, 0.242 mmol) was added EDC.HCl (69.5 mg, 0.363 mmol), HOBt (65.36 mg, 0.484 mmol), then stirred at room temperature for 3 hours, followed by adding methyl 4-(aminomethyl)benzoate hydrochloride (0.24 g, 1.21 mmol) in 1mL NaHCO₃. The mixture was stirred at room temperature overnight. The solvent was evaporated and the material purified by prep-HPLC (octyl column) to give the titled compound.

¹H NMR (CDCl₃) □ 7.65 (d, 2H, J=7.9 Hz), 7.27 (d, 1H, J=8.9 Hz), 7.02 (d, 2H, J=8.0 Hz), 6.75 (d, 1H, J=8.9 Hz), 5.95 (brd, 1H), 4.24 (d, 2H, J=5.8 Hz), 3.87 (t, 2H, J=6.0 Hz), 2.59 (t, 2H, J=7.5 Hz), 2.28 (t, 2H, J=7.3 Hz), 1.96 (pent, 2H, J=6.6 Hz), 1.39 (sext, 2H, J=7.4 Hz), 0.95 (t, 3H, J=7.4 Hz).

MS: m/z=479 (M+H)

### EXAMPLE 19

### Preparation of N-(4-carboxyphenyl)methyl 4-{[7-propyl-3-(trifluromethyl)-1,2-benzisoxazol-6-yl]oxy}butyramide

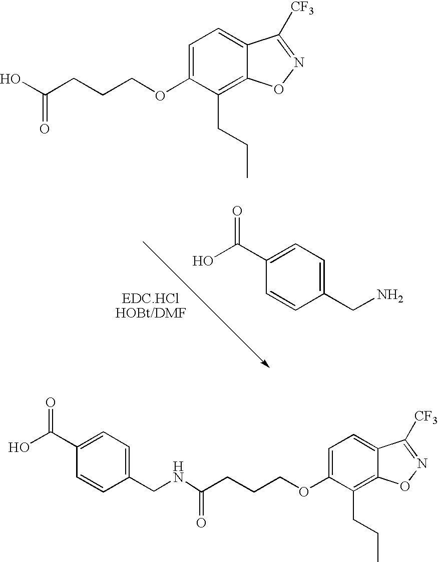

### Preparation of

To a DMF solution (2.0 mL) of 4-{[7-propyl-3-(trifluoromethyl)-1,2-benzisoxazol-6-yl]oxy}butyric acid from Example 5 Step 2 (0.11 g, 0.33 mmol) was added EDC.HCl (95.65 mg, 0.50 mmol), HOBt (89.86 mg, 0.66 mmol), then stirred at room temperature for 3 hours, followed by adding 4-(aminomethyl) benzoic acid (0.177 g, 1.66 mmol) in 2 mL NaHCO₃(saturated). The mixture was stirred at room temperature overnight. The solvent was evaporated and the material purified by prep-HPLC (octyl column) to give the titled compound.

¹H NMR partial (CDCl₃) □ 7.99 (d, 2H, J=8.3 Hz), 7.55 (d, 1H, J=8.9 Hz), 7.34 (d, 2H, J=8.3 Hz), 7.04 (d, 2H, J=8.7 Hz), 5.82 (brd, 1H), 4.54 (d, 2H, J=6.0 Hz), 4.17 (t, 2H, J=6.0 Hz), 2.89 (t, 2H, J=7.4 Hz), 2.51 (t, 2H, J=7.1 Hz), 2.23 (pent, 2H, J=6.7 Hz), 0.95 (t, 3H, J=7.4 Hz).

MS: m/z=465 (M+H)

### EXAMPLE 20

### Preparation of N-Methyl-N-(4-carboxyphenyl)methyl 4-{[7-propyl-3-(trifluromethyl)-1,2-benzisoxazol-6-yl]oxy}butyramide

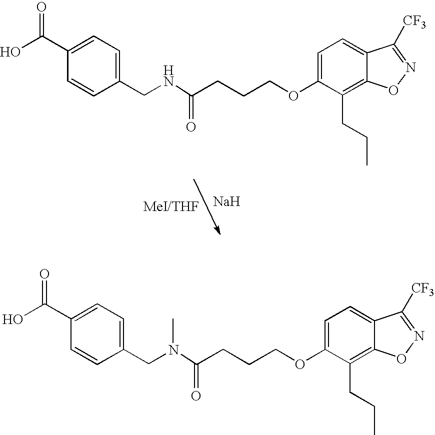

### Preparation of

To a THF solution (2.0 mL) of benzoic acid from Example 19 above (32.0 mg, 0.07 mmol) was added NaH (5.6 mg, 0.14 mmol) and MeI (13.0 uL, 0.21 mmol), then stirred at 40° C. for 5 hours. The solvent was evaporated and the material purified by prep—HPLC (octyl column) to give the titled compound.

¹H NMR two rotamers observed, major reported for most peaks (CDCl₃) □ 8.04 (d, 2H, J=8.3 Hz), 7.57 (d, 1H, J=8.7 Hz), 7.33 (d, 2H, J=8.3 Hz), 7.06 (d, 1H, J=9 Hz), 4.70 (s, 2H), 4.21 (t, 2H, J=5.8 Hz), 3.04 (s, 2H minor), 3.02 (s, 2H major), 2.91 (t, 2H, J=7.4 Hz), 2.72 (m), 2.27 (m), 1.69 (sext, 2H, J=7.4 Hz), 0.95 (t, 3H, J=7.4 Hz major), 0.843 (t, 3H, J=7.3 Hz minor rotamer).

MS: m/z=479 (M+H)

### EXAMPLE 21

### Preparation of N-(3-carbo-t-butyloxyphenyl)methyl 4-{[7-propyl-3-(trifluromethyl)-1 2-benzisoxazol-6-yl]oxy}butyramide

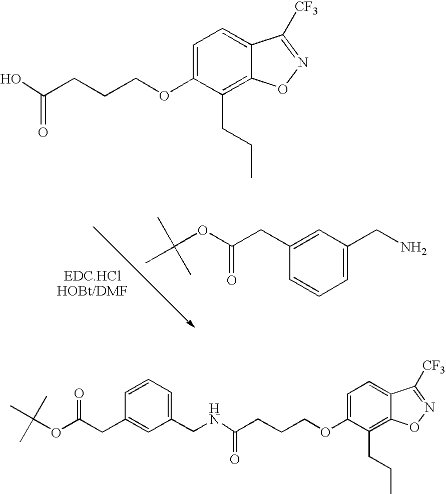

To a DMF solution (2.0 mL) of 4-{[7-propyl-3-(trifluoromethyl)-1,2-benzisoxazol-6-yl]oxy}butyric acid from Example 5 Step 2 (80.0 mg, 0.24 mmol) was added EDC.HCl (69.5 mg, 0.36 mmol), HOBt (65.4 mg, 0.48 mmol), then stirred at room temperature for 3 hours, followed by adding 3-(aminomethyl) phenyl acetic t-Bu ester (0.26 g, 1.2 mmol) in 1 mL DMF. The mixture was stirred at room temperature overnight. After aqueous work-up (ethyl acetate) and chromatography on silica gel using ethyl acetate and hexane (6:4) the titled compound was obtained.

MS: m/z=535 (M+H)

### Step 2 Preparation of N-(3-carboxyphenyl)methyl 4-{[7-propyl-3-(trifluromethyl)-1,2-benzisoxazol-6-yl]oxy}butyramide

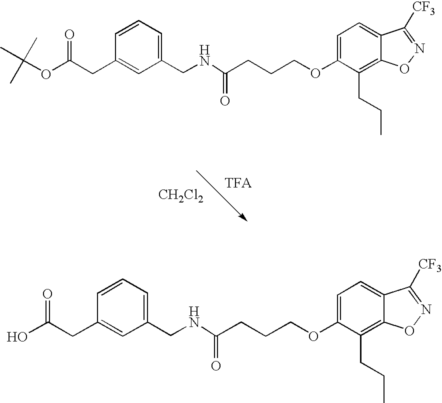

To a CH₂Cl₂ solution of t-Bu ester (1.0 mL) from step 1 above (0.1 g, 0.19 mmol) was added TFA (0.4 mL), then stirred at room temperature for 4 hours. The solvent was evaporated and the material purified by prep-HPLC (octyl column) to give the titled compound.

¹H NMR All resonances broadened due to hindered rotation. (CDCl₃) □ 10.4 (brd, 1H), 7.55 (d, 1H, J=8.7 Hz), 7.24 (m, 1H), 7.16 (m, 3H), 7.03 (d, 2H, J=8.7 Hz), 6.23 (brd, 1H), 4.4 (m, 2H), 4.13 (m, 2H), 3.57 (m, 2H), 2.88 (m, 2H), 2.48 (m, 2H), 2.22 (m, 2H), 1.67 (sext, 2H, J=7.1 Hz), 0.94 (t, 3H, J=7.3 Hz).

MS: m/z=480 (M+H)

### Step 3 Preparation of N-Methy;1-N-(3-carboxyphenyl)methyl 4-{[7-propyl-3-(trifluromethyl)-1,2-benzisoxazol-6-yl]oxy}butyramide

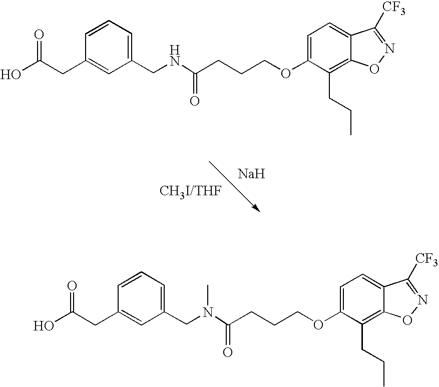

To a THF solution (2.0 mL) of the acid from step 2 above(47.0 mg, 0.10 mmol) was added NaH (8.1 mg, 0.21 mmol) and MeI (19.0 uL, 0.30 mmol), then heated at 40° C. for 3 hours. The solvent was evaporated and the material purified by prep-HPLC (octyl column) to give the titled compound.

¹H NMR All resonances doubled due to hindered rotation. (CDCl₃) □ 10.2 (brd, 1H), 7.55 (2 doublets, 1H), 7.3-7.0 (multiplets, 5H), 4.63 (s, 2H, major), 4.61 (s, 2H minor), 4.17 (m, 2H), 3.64 (2 singlets, 2H), 3.03 (s, 3H, major), 3.01 (s, 3H, minor), 2.88 (m), 2.74 (m), 2.24 (m, 2H), 1.69 (sext, major), 1.58 (sext, minor), 0.94 (t, 3H, J=7.3 Hz major), 0.84 (t, 3H, J=7.3 Hz minor).

MS: m/z=493 (M+H)

### EXAMPLE 22

### Preparation of N-(2-(carbo-t-butyloxy)methylphenyl)methyl 4-{[7-propyl-3-(trifluromethyl)-1,2-benzisoxazol-6-yl]oxy}butyramide

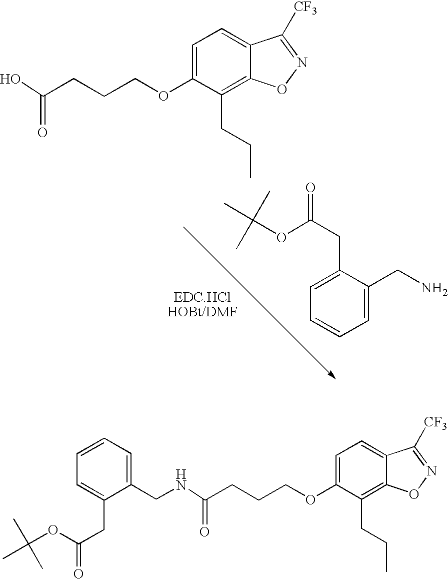

To a DMF solution (2.0 mL) of 4-{[7-propyl-3-(trifluoromethyl)-1,2-benzisoxazol-6-yl]oxy}butyric acid from Example 5 Step 2 (80.0 mg, 0.24 mmol) was added EDC.HCl (69.5 mg, 0.36 mmol), HOBt (65.4 mg, 0.48 mmol), then stirred at room temperature for 3 hours, followed by adding 2-(aminomethyl) phenyl acetic t-Bu ester (0.26 g, 1.2 mmol) in 1 mL DMF. The mixture was stirred at room temperature overnight. After aqueous work-up (ethyl acetate) and chromatography on silica gel using ethyl acetate and hexane (6:4) the titled compound was obtained.

MS: m/z=535 (M+H)

### Step 2 Preparation of N-[2-(carboxymethyl)phenyl]methyl 4-{[7-propyl-3-(trifluromethyl)-1,2-benzisoxazol-6-yl]oxy}butyramide

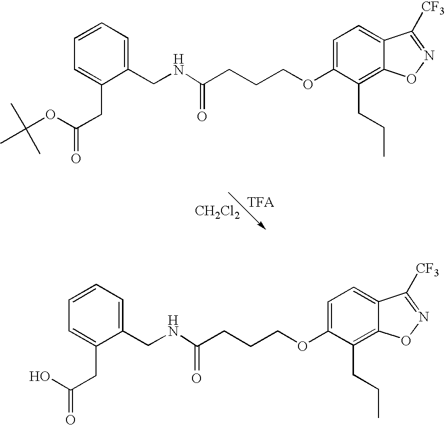

To a CH₂Cl₂ solution of t-Bu ester (1.0 mL) from step 1 above (31.9 mg, 0.06 mmol) was added TFA (0.2 mL), then stirred at room temperature for 4 hours. The solvent was evaporated and the material purified by prep-HPLC (octyl column) to give the titled compound.

¹H NMR All resonances doubled due to hindered rotation. (CDCl₃) □ 7.53 (overlapping d, 1H), 7.1-7.0 (m, 5H), 7.05 (d, 1H, J=9.0 Hz, major), 7.01 (d, 2H, J=9.0 Hz minor), 6.38 (brd, 1H), 6.07 (brd, 1H), 4.46 (d, 2H, J=5.3 Hz minor), 4.42 (d, 2H, J=5.7 Hz major), 4.14 (t, 2H, J=6.1 Hz major), 4.10 (t, 2H, J=6.0 Hz minor), 3.70 (s, 2H minor), 3.58 (s, 2H major), 2.88 (m, 2H), 2.48 (t, 2H, J=7.2 Hz, major), 2.42 (t, 2H, J=7.2 Hz, minor), 2.22 (m, 2H), 1.67 (m, 2H), 0.94 (overlapping t, 3H).

MS: m/z=480 (M+H)

### Step 3 Preparation of N-Methyl-N-[2-(carboxymethyl)phenyl]methyl 4-{[7-propyl-3-(trifluromethyl)-1,2-benzisoxazol-6-yl]oxy}butyramide

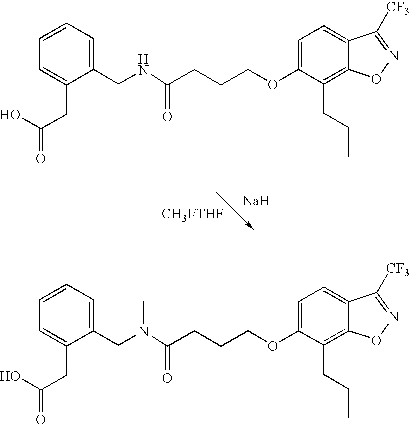

To a THF solution (2.0 mL) of acid from step 2 above (16.0 mg, 0.035 mmol) was added NaH (3.0 mg, 0.07 mmol) and MeI (6.5 uL, 0.10 mmol), then heated at 40° C. for 3 hours. The solvent was evaporated and the material purified by prep-HPLC (octyl column) to give the titled compound.

MS: m/z=493 (M+H)

### EXAMPLE 23

### Preparation of t-Butyl ester of 4-{[7-propyl-3-(trifluromethyl)-1,2-benzisoxazol-6-yl]oxy}butyric acid valine amide

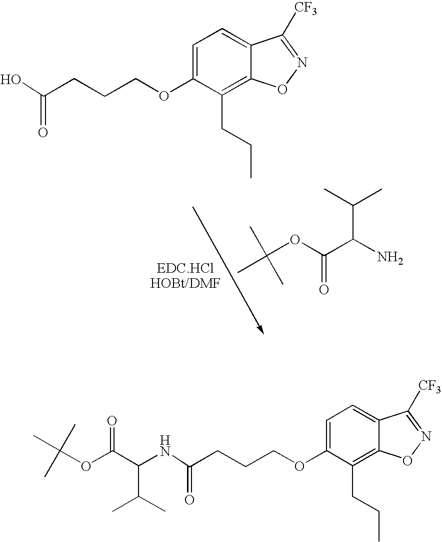

To a DMF solution (1.0 mL) of 4-{[7-propyl-3-(trifluoromethyl)-1,2-benzisoxazol-6-yl]oxy}butyric acid from Example 5 Step 2 (80.0 mg, 0.242 mmol) was added EDC.HCl (69.55 mg, 0.3630 mmol), HOBt (65.35 mg, 0.48 mmol ), then stirred at room temperature for 3 hours, followed by adding H-VAL-OTBU (0.21 g, 1.21 mmol) in 1 mL DMA. The mixture was stirred at room temperature overnight. The solvent was evaporated and chromatography on silica gel using ethyl acetate and hexane (3:7) the titled compound was obtained.

MS: m/z=487 (M+H)

### EXAMPLE 24

### Step 1 Preparation of rac 4-{[7-propyl-3-(trifluromethyl)-1,2-benzisoxazol-6-yl]oxy}butyric acid valine amide

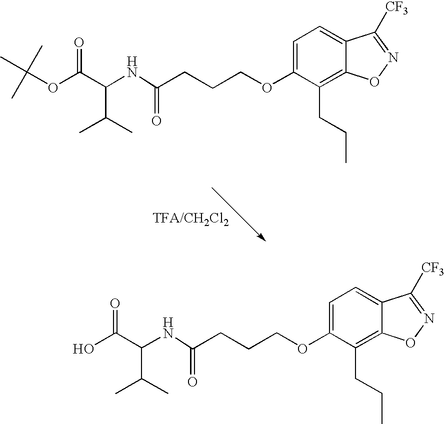

Solution of t-Bu ester (1.0 mL CH₂Cl₂) from Example 23 above (0.07 g, 0.144 mmol) was added TFA (0.4 mL), then stirred at room temperature for 4 hours. The solvent was evaporated and the material purified by prep-HPLC (octyl column) to give the titled compound.

¹H NMR (CDCl₃) □ 7.55 (d, 1H, J=8.9 Hz), 7.05 (d, 1H, J=8.7 Hz), 5.92 (d, 1H), 4.60 (dd, 1H, J=8.7, 4.8 Hz), 4.17 (dt, 1H, J obscured), 2.93 (t, 2H, J=7.5 Hz), 2.53 (t, 2H, J=7.2 Hz), 2.22 (m, 2H), 1.69 (sext, 2H, J=7.5 Hz), 0.98 (overlapping t and d, 6H), 0.94 (t, 3H, J=7.3 Hz).

MS: m/z=431 (M+H)

### Step 2 Preparation of rac 4-{[7-propyl-3-(trifluromethyl)-1,2-benzisoxazol-6-yl]oxy}butyric acid N-mehtylvaline amide

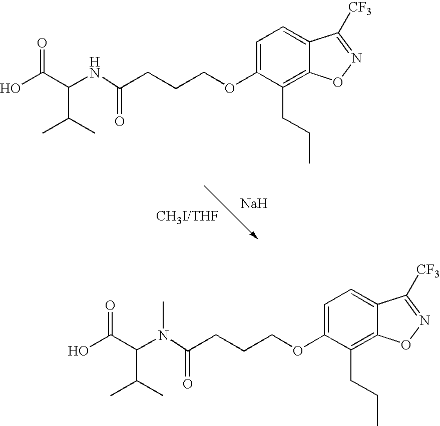

To a solution (2.0 mL) of the acid from step 1 above (0.0392 g, 0.09 mmol) was added NaH (73.0 mg, 0.182 mmol) and MeI (17 uL, 0.274 mmol), then stirred at 40° C. for 5 hours. The solvent was evaporated and the material purified by prep-HPLC (octyl column) to give the titled compound.

¹H NMR Most resonances doubled due to hindered rotation. (CDCl₃) □ 7.55 (d, 1H, J=8.7 Hz), 7.06 (d, 1H, J=8.7 Hz), 4.69 (brd d, 1H), 4.17 (m, 2H), 3.04 (s, 3H), 2.89 (m, 2H), 2.62 (t, 2H, J=7.1 Hz), 2.22 (m, 3H1), 1.69 (sext, 2H, J=7.5 Hz), 1.05 (d, 3H, J=6.7 Hz), 0.95 (t, 3H, J=7.3 Hz), 0.87 (d, 3H, J=6.7 Hz).

MS: m/z=445 (M+H)

### EXAMPLE 25

### Preparation of N-Methyl-N-(4-pyridyl) 4-{[7-propyl-3-(trifluromethyl)-1,2-benzisoxazol-6-yl]oxy}butyramide

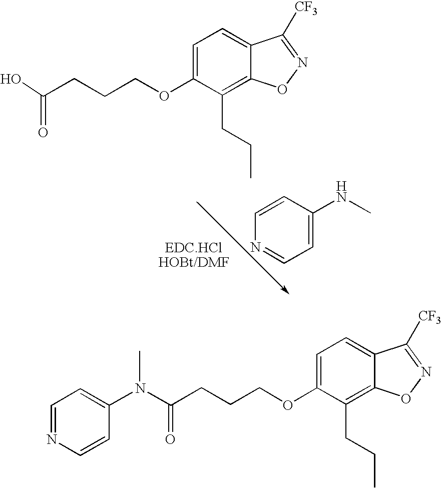

To a DMP solution (1.0 mL) of 4-{[7-propyl-3-(trifluoromethyl)-1,2-benzisoxazol-6-yl]oxy}butyric acid from Example 5 Step 2 (80.0 mg, 0.24 mmol) was added EDC.HCl (69.5 mg, 0.36 mmol), HOBt (65.4 mg, 0.48 mmol), then stirred at room temperature for 3 hours, followed by adding 4-N-methyl pyridine (78.4 mg, 0.73 mmol) in 1 mL DMF. The mixture was stirred at room temperature overnight. After aqueous work-up (ethyl acetate) and chromatography on silica gel using methanol and methylene chloride (1:19) the titled compound was obtained.

¹H NMR (CDCl₃) □ 8.67 (d, 2H, J=5.9 Hz), 7.55 (d, 1H, J=9.0 Hz), 7.19 (d, 2H, J=5.5 Hz), 7.04 (d, 1H, J=9.0 Hz), 4.15 (t, 1H, J=6.0 Hz), 3.36 (s, 3H), 2.82 (t, 2H, J=7.3 Hz), 2.51 (m, 2H), 1.63 (sext, 2H, J=7.3 Hz), 0.906 (t, 3H, J=7.3 Hz).

MS: m/z=422 (M+H)

### EXAMPLE 26

### Preparation of N-Methyl-N-(2-pyridyl) 4-{[7-propyl-3-(trifluromethyl)-1,2-benzisoxazol-6-yl]oxy}butyramide

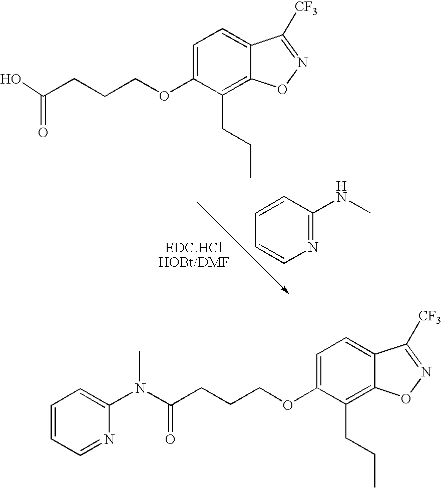

To a DMF solution (1.0 mL) of 4-{[7-propyl-3-(trifluoromethyl)-1,2-benzisoxazol-6-yl]oxy}butyric acid from Example 5 Step 2 (80.0 mg, 0.24 mmol) was added EDC.HCl (69.5 mg, 0.36 mmol), HOBt (65.4 mg, 0.48 mmol ), then stirred at room temperature for 3 hours, followed by adding 2-N-methyl pyridine (75 uL, 0.73 mmol) in 1 mL DMF. The mixture was stirred at room temperature overnight. After aqueous work-up (ethyl acetate) and chromatography on silica gel using methanol and methylene chloride (1:19) the titled compound was obtained.

¹H NMR (CDCl₃) □ 8.48 (m, 1H), 7.78 (m, 1H), 7.55 (d, 1H, J=8.7 Hz), 7.23 (m, 1H), 7.07 (d, 1H, J=8.9 Hz), 4.15 (t, 1H, J=6.0 Hz), 3.41 (s, 3H), 2.82 (t, 2H, J=7.5 Hz), 2.56 (m, 2H), 2.22 (m, 2H), 1.61 (sext, 2H, J=7.4 Hz), 0.900 (t, 3H, J=7.3 Hz).

MS: m/z=422 (M+H)

### EXAMPLE 27

### Step 1 Preparation of N-(4-{[7-propyl-3-(trifluoromethyl)-1,2-benzisoxazol-6-yl]oxy}butanoyl)-L-alanine-t-butyl ester

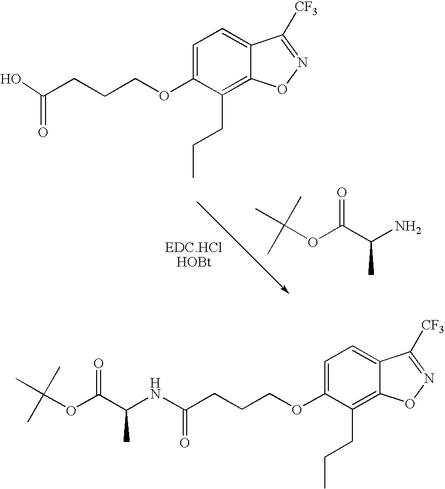

To a CH₂Cl₂ solution (1 mL) of 4-{[7-propyl-3-(trifluoromethyl)-1,2-benzisoxazol-6-yl]oxy}butyric acid from Example 5 Step 2 (80 mg, 0.24 mmol) was added EDC.HCl (69.5 mg, 0.36 mmol) and HOBt (65.4 mg, 0.48 mmol). The mixture was stirred at room temperature for 3 hour, then L-alanine-t-butyl ester (199.4 mg, 1.21 mmol) was added. The reaction mixture was stirred at room temperature overnight. The solvent was evaporated and the material was purified by chromatography on silica gel using ethyl acetate and hexane (3:7) to give the titled compound.

¹H NMR (CDCl₃) □ 7.54 (d, 1H, J=8.5 Hz), 7.07 (d, 1H, J=8.5), 6.05 (d, 1H, J=7.5), 4.48 (m, 1H), 4.16 (m, 2H), 2.91 (t, 2H, J=7.5), 2.44 (t, 2H, J=7.5), 2.20 (m, 2H), 1.72 (m, 2H), 1.46 (s, 9H), 1.37 (d, 3H, J=7.0), 0.97 (t, 3H, J=7.5).

MS: m/z=459 (M+H)

### Step 2 Preparation of N-(4-{[7-propyl-3-(trifluoromethyl)-1,2-benzisoxazol-6-yl]oxy}butanoyl)-L-alanine

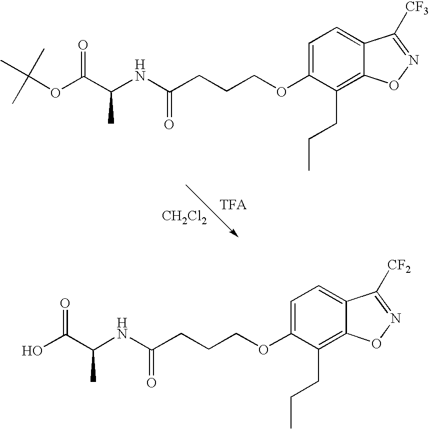

To a CH₂Cl₂ solution (1 mL) of N-(4-{[7-propyl-3-(trifluoromethyl)-1,2-benzisoxazol-6-yl]oxy}butanoyl)-L-alanine-t-butyl ester (70mg, 0.15 mmol) was added TFA (0.4 mL) and the reaction mixture was stirred at room temperature overnight. The solvent was evaporated and the material purified by prep-HPLC (octyl column) to give the titled compound.

¹H NMR (CDCl₃) □ 7.55 (d, 1H, J=9.0 Hz), 7.06 (d, 1H, J=9.0), 5.97 (d, 1H, J=7.0), 4.61 (m, 1H), 4.16 (t, 2H, J=6.0), 2.91 (t, 2H, J=7.5), 2.50 (t, J=7.0), 2.22 (m, 2H), 1.72 (m, 2H), 1.46 ( (d, 3H, J=7.5), 0.97 (t, 3H, J=7.5).

MS: m/z=403 (M+H)

### Step 3 N-methyl-N-(4-{[7-propyl-3-(trifluoromethyl)-1,2-benzisoxazol-6-yl]oxy}butanoyl)-L-alanine

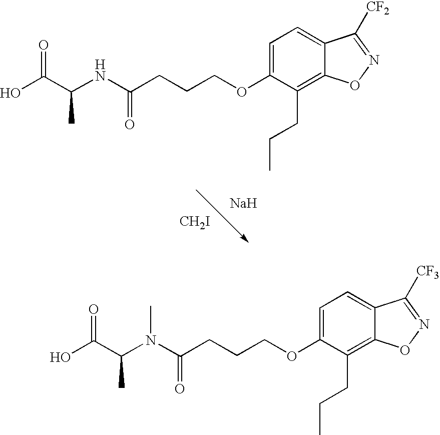

To a THF solution (1 mL) of N-(4-{[7-propyl-3-(trifluoromethyl)-1,2-benzisoxazol-6-yl]oxy}butanoyl)-L-alanine (34.5 mg, 0.09 mmol) was added NaH (7.0 mg, 0.17 mmol) and the reaction mixture was stirred at 40° C. for 1 hour, then MeI (17 □L, 0.26 mmol) was added and stirred at 40° for 2 hours. At this point another MeI (8 □L, 0.13 mmol) was added and stirred at 40° C. for a further 1 hour. The solvent was evaporated and the material purified by prep-HPLC (octyl column) to give the titled compound.

¹H NMR (CDCl₃) □ 10.32 (bs, 1H),7.53 (d, 1H, J=8.5 Hz), 7.06 (d, 1H, J=9.0), 5.17 & 4.62 (q, 1H, J=7.5 and 14.5), 4.16 (t, 2H, J=5.5), 3.00 (s, 3H), 2.89 (t, 2H, J=7.5), 2.63 (m, 2H), 2.21 (m, 2H), 1.69 (m, 2H), 1.49 & 1.43 ((d, 3H, J=7.0), 0.965(t, 3H, J=7.0).

MS: m/z=417 (M+H)

While the invention has been described and illustrated with reference to certain particular embodiments thereof, those skilled in the art will appreciate that various changes, modifications and substitutions can be made therein without departing from the spirit and scope of the invention. For example, effective dosages other than the particular dosages as set forth herein above may be applicable as a consequence of variations in the responsiveness of the mammal being treated for any of the indications for the active agents used in the instant invention as indicated above. Likewise, the specific pharmacological responses observed may vary according to and depending upon the particular active compound selected or whether there are present pharmaceutical carriers, as well as the type of formulation employed, and such expected variations or differences in the results are contemplated in accordance with the objects and practices of the present invention. It is intended, therefore, that the invention be defined by the scope of the claims which follow and that such claims be interpreted as broadly as is reasonable.

1. A compound of Formula I 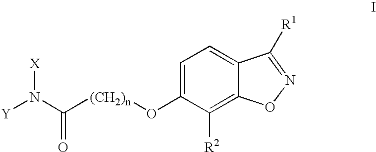
or a pharmaceutically acceptable salt thereof, wherein
R¹ is selected from the group consisting of:
(a) —CF₃,
(b) —C₁₋₆ alkyl, and
(c) —(CH₂)₀₋₂-phenyl;
R² is selected from the group consisting of:
(a) —C₁₋₆ alkyl,
(b) —COOR³,
(c) —CR³R⁴—O—R⁵,
(d) —CR³R⁴—S—R⁵ and
(e) —COR³;
R³, R⁴ and R⁵ are independently selected at each occurrence from the group consisting of —H, phenyl and C₁₋₆ alkyl;
n is an integer selected from 2, 3, 4, 5 and 6;
X is selected from the group consisting of:
(a) —H and
(b) —C₁₋₆ alkyl;
Y is selected from the group consisting of:
(a) —H,
(b) —C₁₋₆ alkyl unsubstituted or substituted with a substituent selected from the group consisting of:
(i) —COOR⁶,
(ii) phenyl, unsubstituted or substituted with —COOR⁶, and
(iii) furanyl,
(c) thiophenyl, unsubstituted or substituted with —COOR⁶, and
(d) pyridinyl, unsubstituted, monosubstituted with a substituent selected from the group consisting of C₁₋₃ alkyl and halogen, or independently disubstituted with two substituents selected from the group consisting of C₁₋₃ alkyl and halogen,
where R⁶ is selected from the group consisting of —H, phenyl and C₁₋₆ alkyl; or Y and X are joined together with the nitrogen to which they are attached to form a piperidinyl ring.

2. A compound of claim 1, or a pharmaceutically acceptable salt thereof, wherein R¹ is selected from the group consisting of CF₃ and C₁₋₆ alkyl, R² is C₁₋₆ alkyl, and n is 3.

3. A compound of claim 2, or a pharmaceutically acceptable salt thereof, wherein,
X is selected from the group consisting of H and C₁₋₃ alkyl, and
Y is selected from the group consisting of:
(a) —H,
(b) —C₁₋₆ alkyl unsubstituted or substituted with a substituent selected from the group consisting of:
(i) —COOR⁶,
(ii) phenyl, unsubstituted or substituted with —COOR⁶, and
(iii) furanyl,
(c) thiophenyl, unsubstituted or substituted with —COOR⁶, and
(d) pyridinyl, unsubstituted, monosubstituted with a substituent selected from the group consisting of C₁₋₃ alkyl and halogen, or independently disubstituted with two substituents selected from the group consisting of C₁₋₃ alkyl and halogen,
where R⁶ is selected from the group consisting of —H, phenyl and C₁₋₆ alkyl; or Y and X are joined together with the nitrogen to which they are attached to form a piperidinyl ring.

4. A compound of claim 3, or a pharmaceutically acceptable salt thereof, wherein R¹ is selected from the group consisting of CF₃ and —CH₂C(CH₃)₃, R² is
—CH₂CH₂CH₃, X is selected from the group consisting of H and —CH₃, Y is selected from the group consisting of 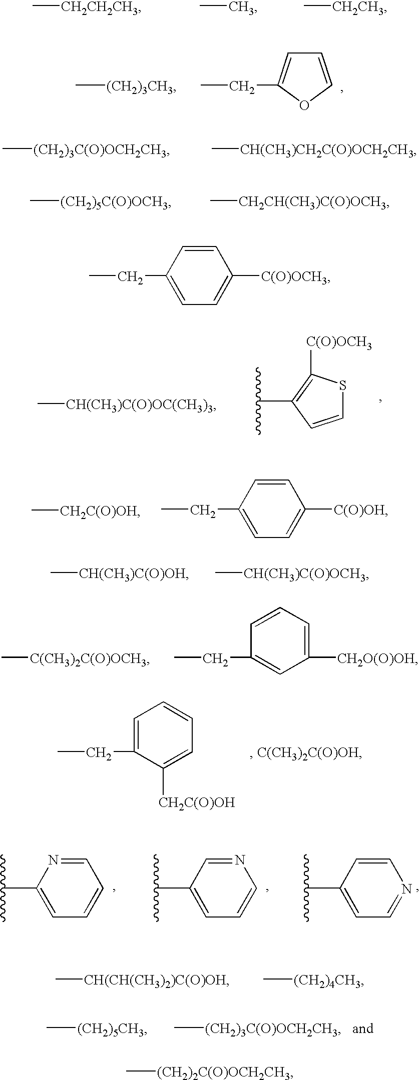
or Y and X are joined together with the nitrogen to which they are attached to form a piperidinyl ring.

5. A compound of claim 4, or a pharmaceutically acceptable salt thereof, having the formula 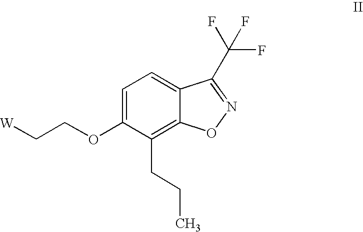
where W is 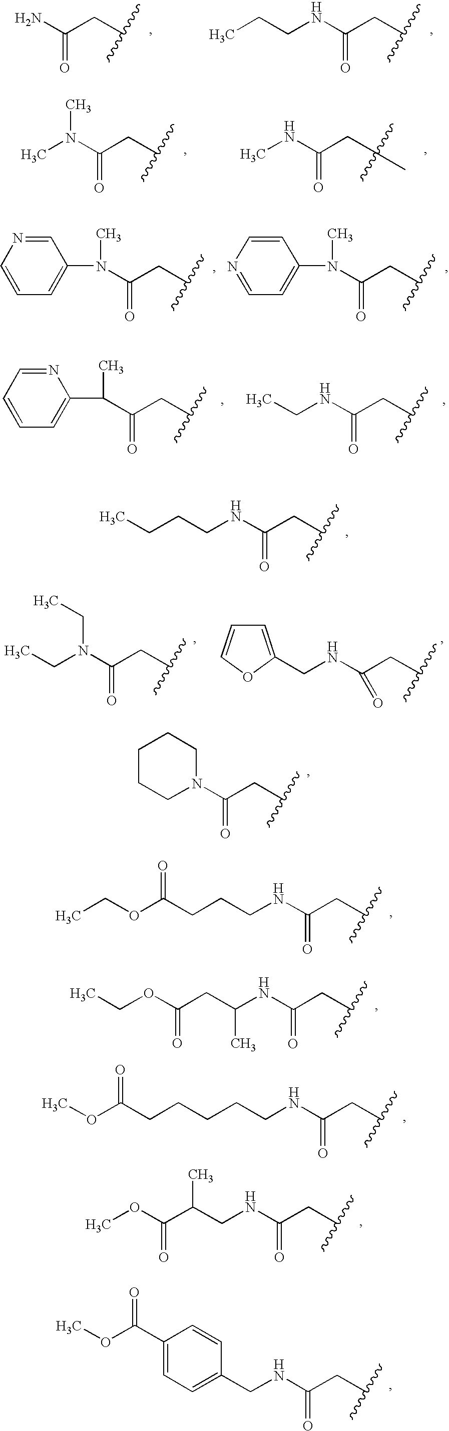 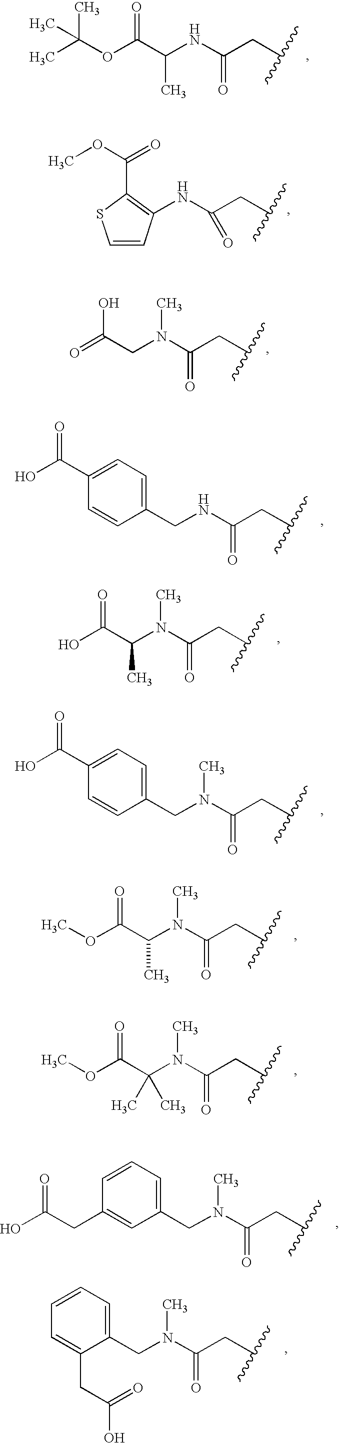 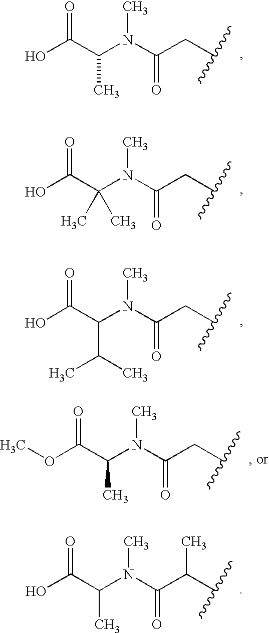
or the formula 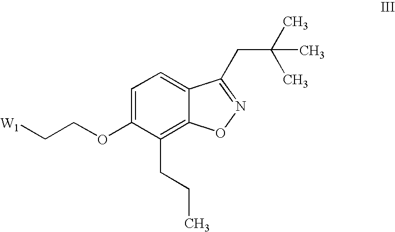
where W₁ is

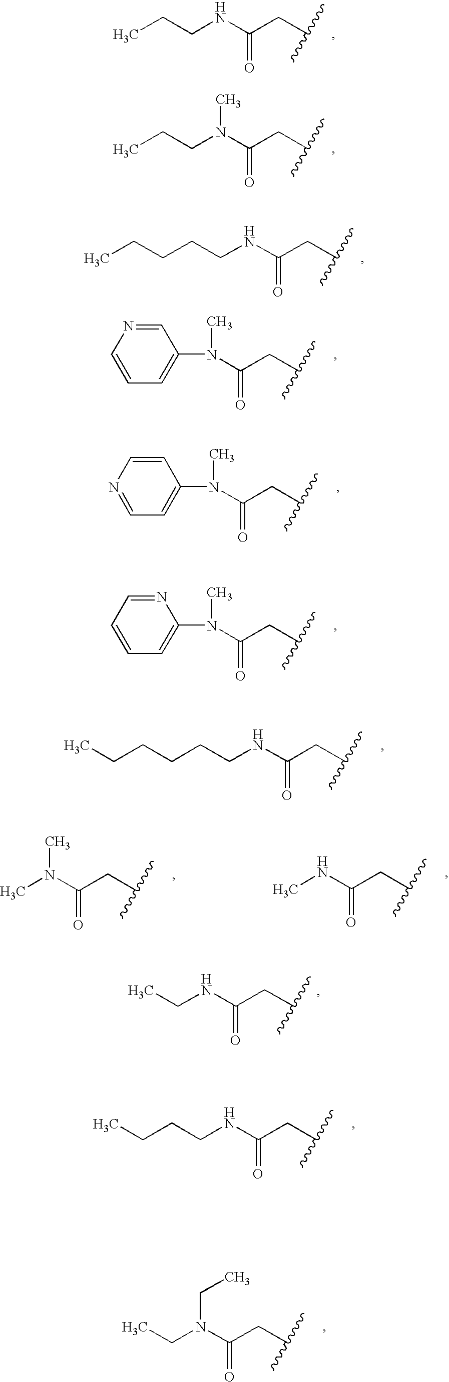

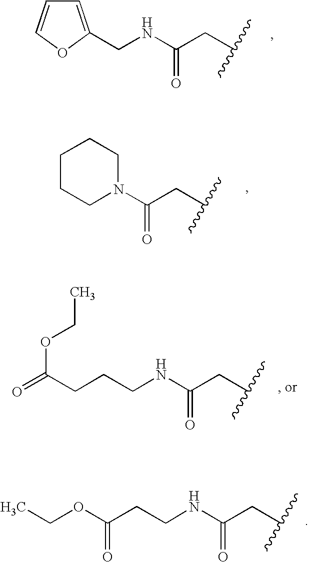

6. A compound of claim 5, or a pharmaceutically acceptable salt thereof, having the formula 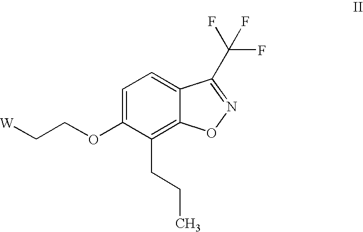
where W is 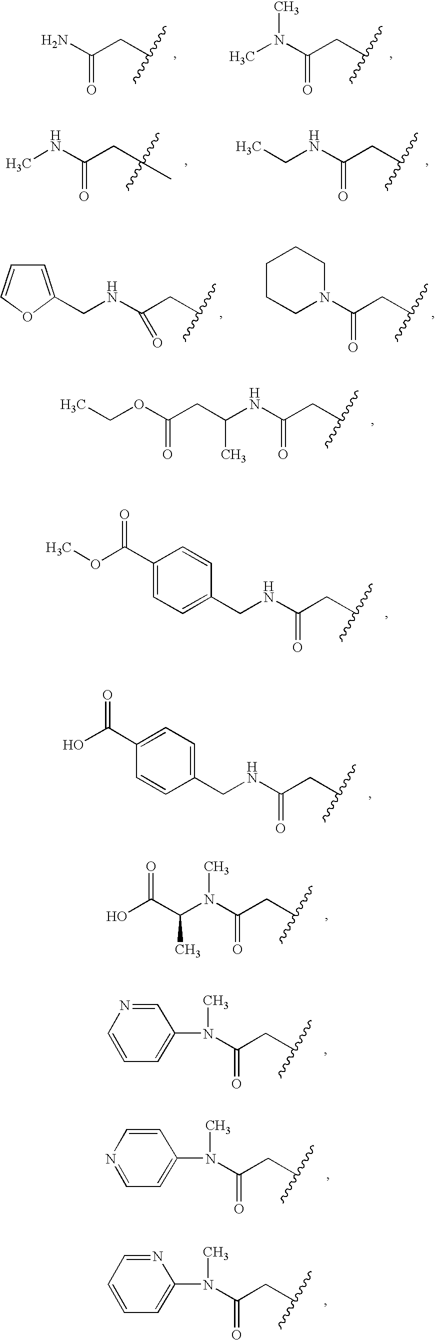 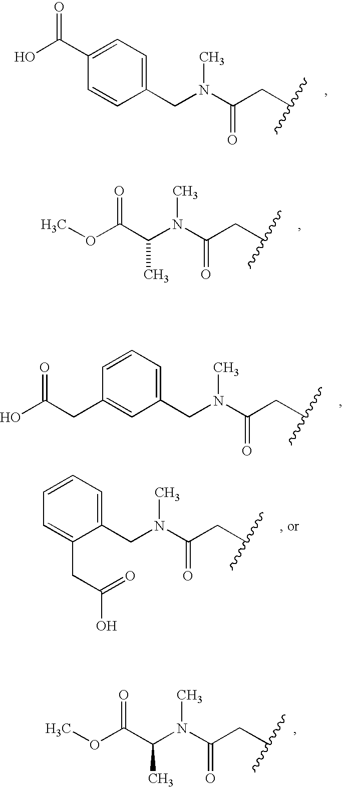
or the formula 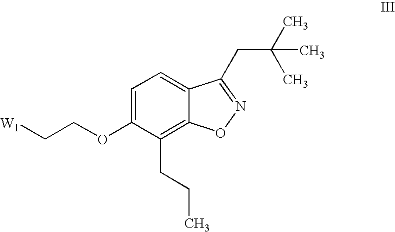
where W₁ is

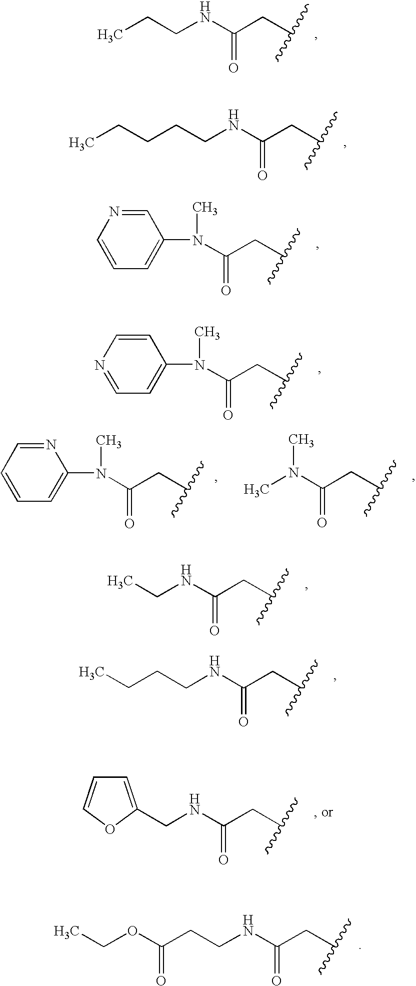

7. A compound of claim 6, or a pharmaceutically acceptable salt thereof, having the formula 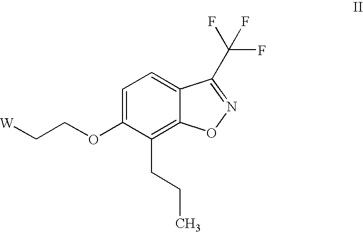
where W is 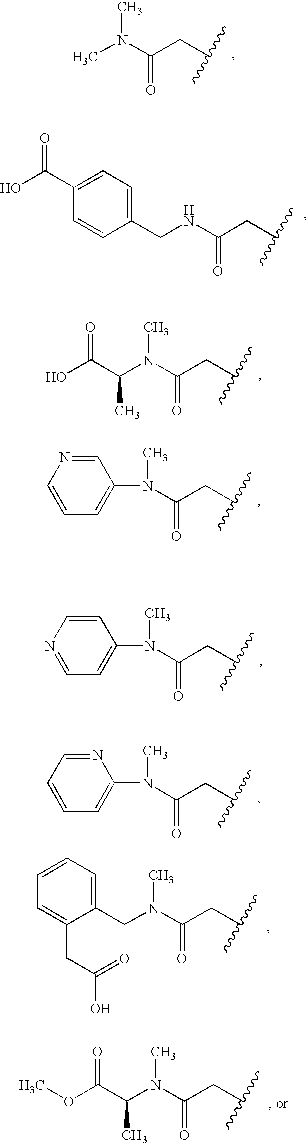
or the formula 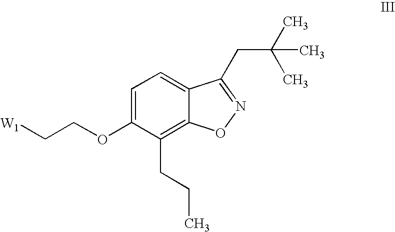
where W₁ is

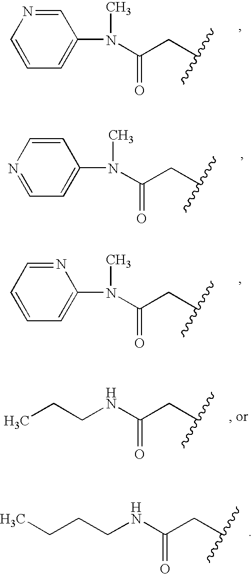

8. A composition comprising a compound of claim 1 and a pharmaceutically acceptable carrier.

9. A method for treating below-desired plasma HDL cholesterol levels in a patient comprising administering to the patient a therapeutically effective amount of a composition of claim 8.

10. A method for treating and/or reducing the risk for diseases and conditions affected by LXR activity in a patient comprising administering to the patient a therapeutically effective amount of a composition of claim 8.

11. A method for preventing lipid accumulation in a patient comprising administering to the patient a therapeutically effective amount of a composition of claim 8.

12. A method for preventing or reducing the risk of developing atherosclerosis in a patient comprising administering to the patient a therapeutically effective amount of a composition of claim 8.

13. A method for preventing or reducing the risk of occurrence of a coronary heart disease event in a patient comprising administering to the patient a therapeutically effective amount of a composition of claim 8.

14. A method of claim 12 further comprising the administration of a prophylactically effective amount of at least one additional agent selected from an HMG-CoA reductase inhibitor, a cyclooxygenase-2 inhibitor, an HMG-CoA synthase inhibitor, a squalene epoxidase inhibitor, a squalene synthetase inhibitor, an ACAT inhibitor, an MFP inhibitor, probucol, niacin, a fibrate, a cholesterol absorption inhibitor, a bile acid sequestrant, an LDL receptor inducer, a platelet aggregation inhibitor, a PPAR agonist, vitamin B₆ and the pharmaceutically acceptable salts thereof, vitamin B₁₂, a beta-blocker, folic acid or a pharmaceutically acceptable salt or ester thereof, vitamin C, vitamin E, beta carotene, a beta-blocker, an angiotensin II antagonist, an angiotensin converting enzyme inhibitor, a calcium channel blocker, an endothelian antagonist, an agent that enhances ABCA1 gene expression, an FXR ligand, a bisphosphonate compound, and an HIV protease inhibitor.

15. The method of claim 14 wherein the HMG-CoA reductase inhibitor is selected from lovastatin, simvastatin, pravastatin, fluvastatin, atorvastatin and the pharmaceutically acceptable salt, ester and lactone forms thereof.

16. The method of claim 15 wherein the HMG-CoA reductase inhibitor is selected from lovastatin and simvastatin.

17. The method of claim 16 wherein the HMG-CoA reductase inhibitor is simvastatin.

18. A pharmaceutical composition of claim 8 further comprising a therapeutically effective amount of at least one additional agent selected from an HMG-CoA reductase inhibitor, a cyclooxygenase-2 inhibitor, an HMG-CoA synthase inhibitor, a squalene epoxidase inhibitor, a squalene synthetase inhibitor, an ACAT inhibitor, an MTP inhibitor, probucol, niacin, a fibrate, a cholesterol absorption inhibitor, a bile acid sequestrant, an LDL receptor inducer, a platelet aggregation inhibitor, a PPAR agonist, vitamin B₆ and the pharmaceutically acceptable salts thereof, vitamin B₁₂, a beta-blocker, folic acid or a pharmaceutically acceptable salt or ester thereof, vitamin C, vitamin E, beta carotene, a beta-blocker, an angiotensin II antagonist, an angiotensin converting enzyme inhibitor, a calcium channel blocker, an endothelian antagonist, an agent that enhances ABCA1 gene expression, an FXR ligand, a bisphosphonate compound, and an HIV protease inhibitor, and a pharmaceutically acceptable carrier.

19. The composition of claim 18 wherein the HMG-CoA reductase inhibitor is selected from lovastatin, simvastatin, pravastatin, fluvastatin, atorvastatin, and the pharmaceutically acceptable salt, ester and lactone forms thereof.

20. The composition of claim 19 wherein the HMG-CoA reductase inhibitor is selected from lovastatin and simvastatin.
